(19) United States
(12) Patent Application Publication
(10) Pub. No.: US 2004/0248850 A1
(43) Pub. Date: Dec. 9, 2004

(54) Compounds for the treatment of HIV infection

(75) Inventors: Justin T. Ernst, San Diego, CA (US); Erik Boman, Bonita, CA (US); Susana C. Ceide, San Diego, CA (US); Antonio G. Montalban, San Diego, CA (US); Hiroshi Nakanishi, San Diego, CA (US); Edward Roberts, San Diego, CA (US); Eddine Saiah, La Jolla, CA (US); Christopher Lum, San Diego, CA (US)

Correspondence Address: FOLEY & LARDNER, P.O. BOX 80278, SAN DIEGO, CA, 92138-0278, US

(73) Assignee: Kemia, Inc.

(21) Appl. No.: 10/774,040

(22) Filed: Feb. 6, 2004

Related U.S. Application Data
(60) Provisional application No. 60/446,713, filed on Feb. 11, 2003.
(60) Provisional application No. 60/523,217, filed on Nov. 18, 2003.

(51) Int. Cl. A61K 31/675; A61K 31/501; A61K 31/4439; C 07D 4 3/02

(52) U.S. Cl. 514/79; 514/89; 514/252.05; 514/341; 544/238; 546/22; 546/268.1; 546/272.7

(57) ABSTRACT

The present invention is related to compounds, their intermediates, processes for their preparation and use, and pharmaceutical compositions comprising the compounds. The novel compounds are useful in therapy, and in particular for the treatment of viral infection, particularly HIV infection.

### CROSS REFERENCES TO RELATED APPLICATIONS

This application claims priority to the U.S. Provisional Application 60/513,217, filed Nov. 18, 2003 and claims priority to U.S. Provisional Application 60/446,713, filed Feb. 11, 2003, the entire contents of which are incorporated herein by reference and for all purposes.

### FIELD OF THE INVENTION

The present invention is related to compounds, intermediates and methods for the preparation and use thereof, and pharmaceutical, compositions comprising the compounds. The novel compounds are useful in antiviral therapy, and in particular for the treatment of HIV infection.

### BACKGROUND OF THE INVENTION

HIV, human immunodeficiency virus, causes acquired immunodeficiency syndrome (AIDS) and while recent advances in drug therapies have been successful in slowing the progression of AIDS, there is still a need to find a safer, more efficient and less expensive way to control this and other viruses. One approach for the development of anti-virals is to inhibit the entry of the virus, such as HIV, into cells. Such an approach should be widely applicable to other viruses, including influenza human respiratory syncytial virus (HRSV), and ebola virus.

For example, the recent elucidation of the HIV entry process has identified many new protein targets for intervention. The function of such proteins is to induce structural changes that allow viral fusion to take place. HIV is an enveloped virus that enters cells by a two step procedure that involves first recognition of receptors on a host cell and then fusion of the viral and host cell membranes. Both of these steps are governed by the envelope protein complex. This complex initially exists as the precursor protein gp160, which is heavily glycosylated and then cleaved by cellular convertase into two subunits: the surface subunit gp120 and the transmembrane subunit gp41. The protein gp41 controls the fusion mechanism of the virus and is activated by gp120 recognition of CD4 receptors and subsequent association of gp120 and a chemokine coreceptor.

Analysis of the entry mechanism of HIV reveals that inhibition of the formation of a critical hexameric helical bundle conformation of gp41 should halt the fusion process. [a) “Peptide and Non-Peptide HIV Fusion Inhibitors,” S. Jiang, Q. Zhao, A. K. Debnath Curr. Pharm. Design 2002, 8, 125-133; b) “HIV-1 Membrane Fusion: Targets of Opportunity,” R. W. Doms, J. P. Moore J. Cell Biol. 2000, 151, F9-F13; c) HIV Fusion and Its Inhibition,” C. C. LaBranche, G. Galasso, J. P. Moore, D. P. Bolognesi, M. S. Hirsch, S. M. Hammer Antiviral Res. 2001, 90, 95-115; d) “HIV-1 Entry—An Expanding Portal for Drug Discovery,” W. S. Blair, P-F. Lin, N. A. Meanwell, O. B. Wallace Drug Discovery Today 2000, 5, 183-194; e) “Development of HIV Entry Inhibitors Targeted to the Coiled-Coil Regions of gp41,” S. Jiang, A. K. Debnath Biochem. Biophys. Res. Commun. 2000, 269, 641-646; f) “HIV Entry and Its Inhibition,” D. C. Chan, P. S. Kim Cell 1998, 93, 681-684.] The hypothesis of six-helix bundle antagonism has been validated by the fact that peptides with sequences derived from gp41 are potent inhibitors of viral fusion. For example, Wild et al. has shown that a peptide, DP-178 (T20), is a potent antiviral agent, with an EC₅₀ of 1 ng/mL in cell culture. [“Peptides Corresponding to a Predictive α-Helical Domain of Human Immunodeficiency Virus Type 1 gp41 are Potent Inhibitors of Virus Infection,” C. T. Wild, D. C. Shugars, T. K. Greenwell, C. B. McDanal, T. J. Matthews Proc. Natl. Acad. Sci. USA 1994, 91, 9770-9774.] The T20 peptide (also known as Fuzeon) received FDA approval in March 2003 for the treatment of HIV infected individuals who have shown resistance to currently marketed reverse transcriptase and protease inhibitors. However, T20 and other peptide agents are unsuitable for oral administration and suffer from poor pharmacokinetic properties.

Other enveloped viral glycoproteins, such as RSV F and Ebola GP2 proteins, show structural and mechanistic similarities to gp41. [“Mechanisms of Viral Membrane Fusion and Its Inhibition”, D. M. Eckert and P. S. Kim Annu. Rev. Biochem. 2001, 70, 777-810]. Such proteins constitute additional targets for inhibition of viral entry into cells.

Therefore a need exists in the art to identify non-peptidic compounds that antagonize the function of gp41 and related proteins and thus inhibit viral entry into cells. In particular, there is a need for non-peptidic compounds that inhibit HIV entry and infection. The compounds having structures outlined below represent a class of molecules that address these needs and possess other advantages.

### SUMMARY OF THE INVENTION

In various aspects, the present invention relates in part to compounds, including those having Formulas I and II; to intermediates of Formula III; to processes for preparing compounds of Formulas I and II; to compositions for treatment using such compounds; to methods of use and treatment with such compounds; and to methods of identifying subjects in need of such treatments.

Thus, one aspect of the invention provides compounds having a first planar moiety directly or indirectly attached to an acidic moiety, to a hydrophobic planar moiety, and to a second planar moiety bearing one or more non-aryl and non-heteroaryl substituents. The first planar moiety is typically a substituted or unsubstituted 6-member aryl or heteroaryl ring that holds the other moieties in a particular orientation relative to each other. The acidic moiety includes a group with at least one acidic proton, such as a carboxylic acid, a boronic acid, or a tetrazole, or a hydrogen bond donor and/or acceptor such as an amide group. Alternatively, the acidic moiety may include a functionality that may be readily converted in vivo or by chemical synthesis to an acidic moiety, e.g. an ester or a primary alcohol. The hydrophobic moiety is typically a large non-polar moiety that may include two or more rings such as phenyl rings and may be substituted or unsubstituted. The second planar moiety may be any of a variety of substituted aryl or heteroaryl rings with one or more non-aryl and non-heteroaryl substituents.

In another aspect, the invention provides compounds having Formula I:

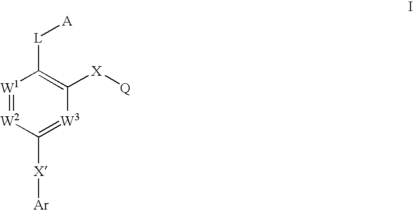

wherein:

A is hydrogen, OH, NO₂, —COOR, —C(O)NROH, —C(O)CF₃, —B(OH)₂, —SO₃H, —PO₃R₂, —OPO₃R₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR;

L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, —NR—C(O)NR—(CR⁴R⁵)ₘ—, —S(O)₂—NR—(CR⁴R⁵)ₘ—, or —NR—S(O)₂—(CR⁴R⁵)ₘ—, provided that L and A together are not H, —CH₃, OH, or —OCH₃.

W¹ is N or CR¹;

W² is N or CR²;

W³ is N or CR³;

X is —(CR⁶R⁷)ᵣ—, —O—(CR⁶R⁷)ᵣ—,—S(O)_{q}—(CR⁶R⁷)ᵣ—, —NR—(CR⁶R⁷)ᵣ—, —NR—C(O)—(CR⁶R⁷)ᵣ—, —C(O)O—(CR⁶R⁷)ᵣ—, —C(O)NR—(CR⁶R⁷)ᵣ—, —NR—C(O)—O(CR⁶R⁷)ᵣ—, —NR—C(O)NR—(CR⁶R⁷)ᵣ—, —S(O)₂—NR—(CR⁶R⁷)ᵣ—, or —NR—S(O)₂—(CR⁶R⁷)ᵣ—;

X′ is a covalent bond, O, S(O)_{q}, —NR—, —N(C(O)—R)—, —N(C(O)—OR)—, —N(C(O)—NRR)—, —NR—C(O)—, —NR—C(O)—NR—, substituted or unsubstituted C₁₋₄ alkyl, substituted or unsubstituted C₂ alkenyl, or acetylenyl;

Q is a substituted or unsubstituted cycloalkyl, substituted or unsubstituted cycloalkenyl, substituted or unsubstituted aryl, substituted or unsubstituted aralkyl, substituted or unsubstituted heterocyclyl, or substituted or unsubstituted heterocyclylalkyl;

Ar is aryl or heterocyclyl, each substituted with one or more R′;

R at each occurrence is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted (C₀₋₄ alkylene)(C₆₋₁₀ aryl), or substituted or unsubstituted (C₀₋₄ alkylene)(C₁₋₉ heterocyclyl);

R′ at each occurrence is independently, F, Cl, Br, I, NO₂, CN, substituted or unsubstituted C₁₋₈ alkyl, substituted or unsubstituted C₂₋₈ alkenyl, substituted or unsubstituted (C₁₋₆ alkylene)(C₆₋₁₄ aryl), substituted or unsubstituted (C₁₋₆ alkylene)(C₁₋₁₃ heterocycl), OR⁸, —C(O)R⁸, —COOR⁸, —S(O)_{q}R⁸, —NR⁸R⁹, —C(Y)NR⁸R⁹, —N(R⁸)C(Y)OR⁹, —NR¹⁰C(Y)NR⁸R⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹, wherein Y is O or S;

R¹, R², and R³, at each occurrence, are independently hydrogen, F, Cl, Br, I, CN, NO₂, substituted or unsubstituted C₁₋₈ alkyl, substituted or unsubstituted C₂₋₈ alkenyl, substituted or unsubstituted (C₀₋₆ alkylene)(C₆₋₁₄ aryl), substituted or unsubstituted (C₀₋₆ alkylene)(C₁₋₁₃ heterocyclyl), OR⁸, —C(O)R⁸,—COOR⁸, —S(O)_{q}R⁸, —NR⁸R⁹, —C(Y′)NR⁸R⁹, —N(R⁸)C(Y′)OR⁹, —NR¹⁰C(Y′)NR⁸R⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹, wherein each Y′ is independently O or S;

R⁴ and R⁵ are, at each occurrence, independently hydrogen, F, Cl, Br, I, substituted or unsubstituted straight or branched C₁₋₄ alkyl, substituted or unsubstituted C₂₋₄ alkenyl, substituted or unsubstituted aryl, substituted or unsubstituted heterocyclyl, —OR, —COOR —NRR; or R⁴ and R⁵, together with the carbon to which they are attached, form a carbonyl;

R⁶ and R¹ are, at each occurrence, independently hydrogen, F, Cl, Br, I, substituted or unsubstituted straight or branched C₁₋₄ alkyl, substituted or unsubstituted C₂₋₄ alkenyl, —OR, —COOR —NRR; or when r is 2 or 3, R⁶ and R⁷, together with the carbon to which they are attached, may form a carbonyl;

R⁸, R⁹, R¹⁰, and R¹¹, at each occurrence, are independently hydrogen, substituted or unsubstituted C₁₋₈ alkyl, substituted or unsubstituted C₂₋₆ alkenyl, substituted or unsubstituted (C₀₋₆ alkylene)(C₆₋₁₀ aryl), or substituted or unsubstituted (C₀₋₆ alkylene)(C₁₋₉ heterocyclyl); or R⁸ and R⁹, together with the N to which they are attached, form a substituted or unsubstituted heterocyclic ring;

m=0-4;

each q is independently 0-2; and

r=0-3;

and stereoisomers thereof, tautomers thereof, solvates thereof, prodrugs thereof, and pharmaceutically acceptable salts thereof.

In some embodiments, the compounds of Formula I do not include acetic acid 3′-(2-acetoxy-4-methoxy-benzoyl)-5-benzoyl-2-methoxy-biphenyl-4-yl ester, acetic acid 5′-(2-acetoxy-4-methoxy-benzoyl)-2,2′-dimethoxy-5-(4-methoxy-benzoyl)-biphenyl-4-yl ester, 5,5′-bis-[bis-(4-tert-butyl-phenyl)-methoxy-methyl]-2,4,2′,4′-tetraisopropyl-biphenyl, 3-acetoxy-5-methyl-2-[2,4,2′,4′-tetraacetoxy-3′-(2-methoxycarbonyl-4-methyl-6-acetoxybenzoyl)-biphenyl-3-carbonyl]-benzoic acid methyl ester, 3-(3-benzyl-4′-methoxy-biphenyl-4-yl)-propionic acid, 3-(3-benzyl-4′-methoxy-biphenyl-4-yl)-propionyl chloride, or (4,4′-diamino-3′-benzoyl-biphenyl-3-yl)-phenyl-methanone.

In some embodiments of compounds having Formula I, A is OH, NO₂, —COOR, —C(O)NROH, —C(O)CF₃, —B(OH)₂, —SO₃H, —PO₃R₂, —OPO₃R₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR. In other embodiments, A is hydrogen, —COOR, —C(O)NROH, —C(O)CF₃, —B(OH)₂, —SO₃H, —PO₃H₂, —OPO₃H₂, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR. In still other embodiments, A is substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR. In yet other embodiments, A is —COOR, —C(O)NHOH, —C(O)CF₃, or —B(OH)₂. In certain embodiments, A is —COOR or —COOH.

In some embodiments of compounds having Formula I, L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—. In other embodiments, L is —(CR⁴R⁵)ₘ—, —O—(CR⁴R⁵)ₘ—, —S(O)_{q}—(CR⁴R⁵)ₘ—, —NR—(CR⁴R⁵)_, —C(O)O—(CR⁴R⁵)ₘ—, or —C(O)NR—(CR⁴R⁵)ₘ—. In still other embodiments, L is —(CR⁴R⁵)ₘ—, —O—(CR⁴R⁵)ₘ—, —S(O)_{q}—(CR⁴R⁵)ₘ—, or —NR—(CR⁴R⁵)ₘ—. In certain embodiments, L is —(CR⁴R⁵)ₘ—, or —O—(CR⁴R⁵)ₘ—, and in others, L is —O—(CR⁴R⁵)ₘ—. In some such embodiments R⁴ and R⁵ are each hydrogen. In other such embodiments, m=1-2. In certain embodiments, L and A together are —(CR⁴R⁵)ₘ—COOR or —O—(CR⁴R⁵)ₘ—COOR. In yet other embodiments, R⁴ and R⁵ are, at each occurrence, independently hydrogen, F, Cl, Br, I, substituted or unsubstituted straight or branched C₁₋₄ alkyl, substituted or unsubstituted C₂₋₄ alkenyl, OR, COOR, —NRR; or R⁴ and R⁵, together with the carbon to which they are attached, form a carbonyl.

As indicated above, the length of the linker L in compounds of Formula I may vary depending upon the choice of the integer, m. In some embodiments, m=1-3 and in others m=1 or 2. Similarly, the length of the linker X may vary depending upon the choice of the integer, r. In some embodiments, r=1-3 or r=1-2. In other embodiments r is 0.

In some embodiments of compounds having Formula I, X is —(CR⁶R⁷)ᵣ—, —O—(CR⁶R⁷)ᵣ—, —S(O)_{q}—(CR⁶R⁷)ᵣ—, —NR—(CR⁶R⁷)ᵣ—, —NR—C(O)—(CR⁶R⁷)ᵣ—, —C(O)O—(CR⁶R⁷)ᵣ—, —C(O)NR—(CR⁶R⁷)ᵣ—, —NR—C(O)—O(CR⁶R⁷)ᵣ—, or —NR—C(O)NR—(CR⁶R⁷)ᵣ—. In other embodiments, X is —(CR⁶R⁷)ᵣ—, —O—(C R⁶R⁷)ᵣ—, —S(O)_{q}—(CR⁶R⁷)ᵣ—, —NR—(CR⁶R⁷)ᵣ—, —C(O)O—(CR⁶R⁷)ᵣ—, or —C(O)NR—(CR⁶R⁷)ᵣ—. In still other embodiments, X is —(CR⁶R⁷)ᵣ—, —O—(CR⁶R⁷)ᵣ—, or —S(O)_{q}—(CR⁶R⁷)ᵣ—. In yet other embodiments, X is —(CR⁶R⁷)ᵣ— and preferably, X is —CH₂—.

In some embodiments of compounds having Formula I, Q is a substituted or unsubstituted cycloalkyl or substituted or unsubstituted cycloalkenyl. In other embodiments, Q is a substituted or unsubstituted aryl, substituted or unsubstituted aralkyl, substituted or unsubstituted heterocyclyl, or substituted or unsubstituted heterocyclylalkyl. In yet other embodiments, Q is a substituted or unsubstituted aryl or substituted or unsubstituted aralkyl. In still other embodiments, Q is a fused or unfused bicyclic ring selected from the group consisting of substituted and unsubstituted C₉₋₁₂ aryl, substituted and unsubstituted C₇₋₁₂ cycloalkyl, substituted and unsubstituted C₉₋₁₂ cycloalkenyl, and substituted and unsubstituted C₇₋₁₂ heterocyclyl. In some embodiments, Q may be a fused or unfused bicyclic ring that is substituted or unsubstituted C₉₋₁₂ aryl, and in particular, may be substituted or unsubstituted 1-naphthyl, 2-naphthyl, or 4-biphenyl. In some such embodiments, X is —CH₂—.

As indicated above in Formula I, the core ring may be attached to Ar in a variety of ways. In some embodiments, X′ is a covalent bond, O, S(O)_{q}, —NR—, —N(C(O)—R)—, —N(C(O)—OR)—, —N(C(O)—NRR)—, or substituted or unsubstituted C₁₋₄ alkyl. In other embodiments, X′ is a covalent bond, O, S(O)_{q}, —NR—, —NR—C(O)—, —NR—C(O)—NR—, substituted or unsubstituted C₁₋₂ alkyl, substituted or unsubstituted C₂ alkenyl, or acetylenyl. In other embodiments, X′ is a covalent bond, O, S(O)_{q}, or —NR—. In still other embodiments, X′ is a covalent bond, O, or —NR—. Typically, X′ is a covalent bond or a substituted or unsubstituted C₁₋₂ alkyl such as —CH₂—. In some other embodiments, X′ may be —N(C(O)—R)—, —N(C(O)—OR)—, or —N(C(O)—NRR)—. In other embodiments, X′ is —N(C(O)—R)—.

A variety of 6-member rings are contemplated to be within the scope of compounds having Formula I. In some embodiments, W¹ is CR¹ and in others, W¹ is N. In some embodiments, W² is CR² and in others, W² is N. In certain embodiments, W³ is CR³, and in others, W³ is N. When W¹ is CR¹, W² is CR², and W³ is CR³, the resulting ring is phenyl substituted by R¹, R² and R³. When one of W¹, W², or W³ is N and the others are CR¹ and CR², the resulting ring is pyridine substituted by R¹ and R². When W¹ is N, W² is N, and W³ is CR³, the resulting ring is pyridazine, substituted by R³. When W¹ is CR¹, W² is N, and W³ is N, the resulting ring is pyrimidine, substituted by R¹. As will be readily recognized by one of skill in the art, various phenyls, pyridines, pyridazines, pyrazines, pyrimidines, and triazines fall within the scope of the present invention as illustrated by structures IA-IH below, wherein A, L, X, X′, Q, Ar, R¹, R², and R³ are as defined herein.

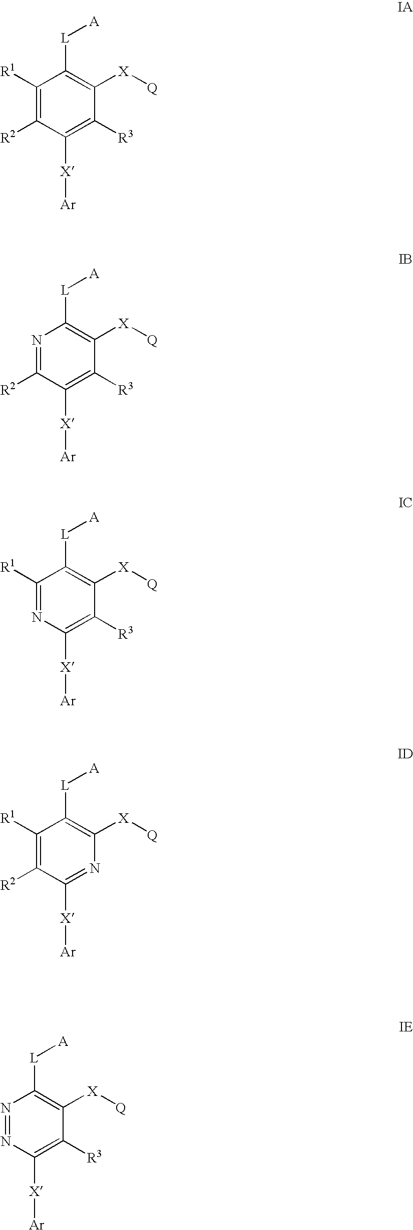

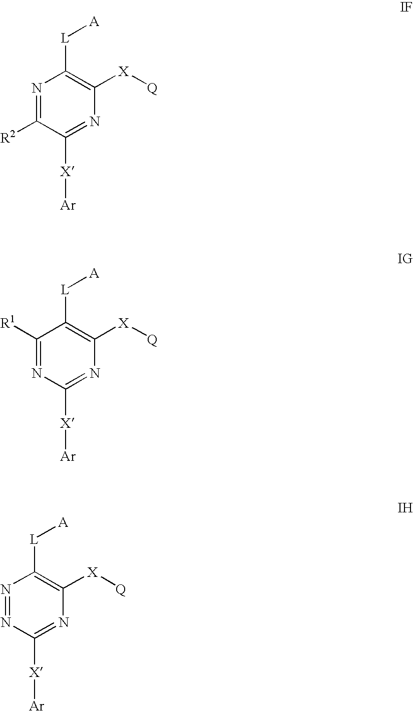

As reflected by Formula I, numerous rings fall within the definition of Ar. In some embodiments, Ar is a 6-member aryl, a 5-or 6-member heteroaryl, a 9-12 member bicyclic aryl or heterocyclyl, each substituted with one or more R′. In some other embodiments, Ar is a 9-12-member bicyclic aryl or heterocyclyl, substituted with one or more R′. In other embodiments, Ar is a 6-member aryl or a 5- or 6-member heteroaryl, substituted with one or more R′. In yet other embodiments, Ar is a 6-member aryl, substituted with one or more R′. In still other embodiments, Ar is a 5- or 6-member heteroaryl, substituted with one or more R′. In other embodiments, Ar is substituted with one or more R′ and is selected from the group consisting of phenyl, pyrrolyl, imidazolyl, pyrazolyl, triazolyl, furanyl, thiophenyl, oxazolyl, isooxazolyl, oxadiazolyl, thiazolyl, isothiazolyl, thiadiazolyl, pyridinyl, pyridazinyl, pyrimidinyl, pyrazinyl, triazinyl and the like. In certain embodiments, Ar is substituted with one or more R′ and is selected from the group consisting of phenyl, pyrrolyl, imidazolyl, pyrazolyl, furanyl, thiophenyl, oxazolyl, isooxazolyl, thiazolyl, isothiazolyl, pyridinyl, pyridazinyl, pyrimidinyl, pyrazinyl, and the like. In yet other embodiments, Ar is substitured with one or more R′ and is selected from the group consisting of naphthyl, indolyl, benzofuranyl, benzthiazolyl, benzothiophenyl, chromanyl, isochromanyl, coumarinyl, and the like. Commonly, Ar is phenyl substituted with one or more R′, to give compounds having Formula II.

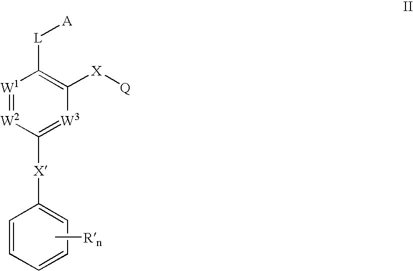

Thus, as those of skill in the art will recognize, when Ar is phenyl, the following structures, IIA-IIH, are contemplated by this aspect of the present invention, wherein A, L, X, Q, X′, R′, R¹, R², and R³ are as defined above, and n=1-5.

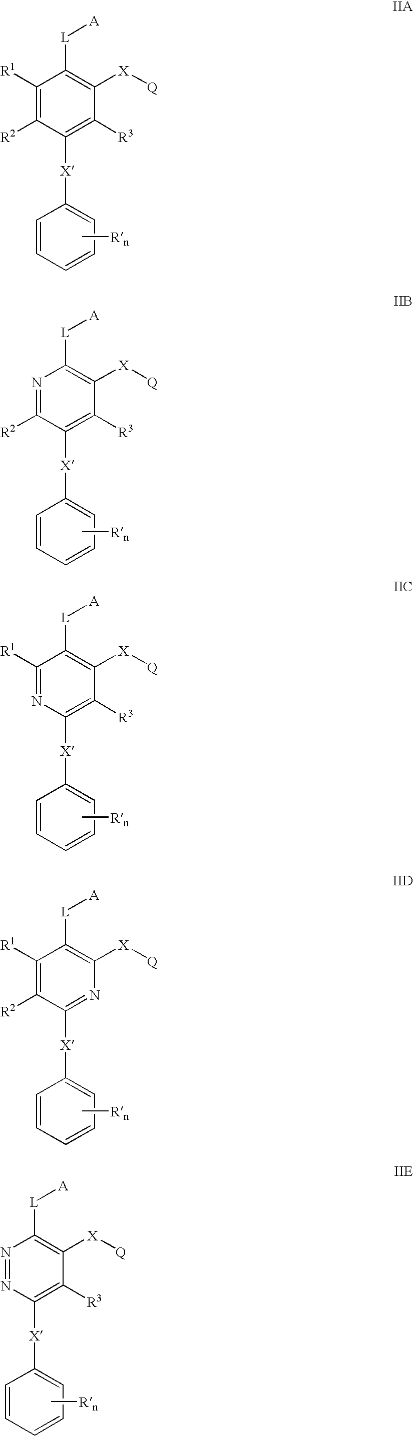

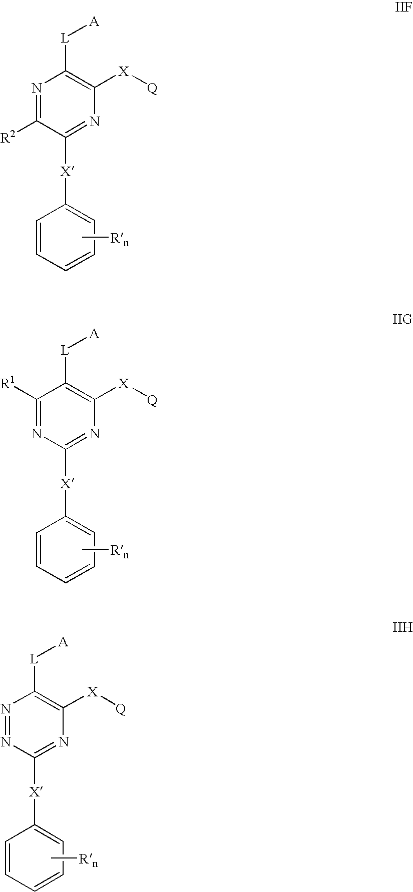

As described herein, compounds of Formula I may have numerous different substituents. In some embodiments, R¹, R², and R³, at each occurrence, are independently hydrogen, F, Cl, Br, I, CN, NO₂, substituted or unsubstituted C₁-C₈ alkyl, substituted or unsubstituted C₂₋₈ alkenyl, substituted or unsubstituted (C₀₋₆ alkylene)(C₆₋₁₄ aryl), substituted or unsubstituted (C₀₋₆ alkylene)(C₁₋₁₃ heterocyclyl), —OR⁸, —C(O)R⁸, —COOR⁸, —S(O)_{q}R⁸, —NR⁸R⁹, —C(O)NR⁸R⁹, —N(R⁸)C(O)OR⁹, —NR¹⁰C(O)NR⁸R⁹, —NR¹⁰C(NR¹¹)NR⁸R⁹, —C(NR¹⁰)NR⁹R⁹, —NR¹⁰NR⁸R⁹, —NR⁸OR⁹, —S(O)_{q}NR⁸R⁹, or —NR⁸—SO₂—R⁹. In other embodiments, R¹, R², and R³, at each occurrence, are independently hydrogen, F, Cl, Br, I, CN, NO₂, substituted or unsubstituted C₁₋₈ alkyl, substituted or unsubstituted C₂₋₈ alkenyl, substituted or unsubstituted (C₀₋₆ alkylene)(C₆₋₁₄ aryl), substituted or unsubstituted (C₀₋₆ alkylene)(C₁₋₁₃ heterocyclyl), —OR⁸, —C(O)R⁸, —COOR⁸, —S(O)_{q}R⁸, —NR⁸R⁹, —C(Y′)NR⁸R⁹, —N(R⁸)C(Y′)OR⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹, wherein each Y′ is independently O or S.

As described herein, compounds of Formula I may have numerous different substituents R′ on Ar. In some embodiments, R′, at each occurrence, is independently, F, Cl, Br, I, CN, NO₂, substituted or unsubstituted C₁₋₈ alkyl, substituted or unsubstituted C₂₋₈ alkenyl, substituted or unsubstituted (C₁₋₆ alkylene)(C₆₋₁₄ aryl), substituted or unsubstituted (C₁₋₆ alkylene)(C₁₋₁₃ heterocyclyl), —OR⁸, —C(O)R⁸, —COOR⁸, —S(O)_{q}R⁸, —NR⁸R⁹, —C(Y)NR⁸R⁹, —N(R⁸)C(Y)OR⁹, —NR¹⁰C(NR¹¹)NR⁸R⁹, —C(NR¹⁰)NR⁸R⁹, —S(O)_{q}NR⁸R⁹, or —NR⁸—SO₂—R⁹, wherein Y is O or S. In yet other embodiments, R′, at each occurrence, is independently F, Cl, Br, I, CN, NO₂, substituted or unsubstituted C₁₋₈ alkyl, substituted or unsubstituted C₂₋₈ alkenyl, substituted or unsubstituted (C₁₋₆ alkylene)(C₆₋₁₄ aryl), substituted or unsubstituted (C₁₋₆ alkylene)(C₁₋₁₃ heterocyclyl), —OR⁸, —C(O)R⁸, —COOR⁸, —NR⁸R⁹, —C(Y)NR⁸R⁹, or —N(R⁸)C(Y)OR⁹, wherein Y is O or S. In other embodiments, R′, at each occurrence, is independently F, Cl, Br, I, NO₂, substituted or unsubstituted C₁₋₈ alkyl, substituted or unsubstituted C₂₋₈ alkenyl, OR⁸, or —COOR⁸. In some embodiments, when L and A together are —O—C(O)—CH₃, X′ is a covalent bond, and Ar is phenyl substituted by —C(O)—R⁸, R⁸ cannot be phenyl substituted with acetoxy. In yet other embodiments, where L and A together are NH₂, X′ is a covalent bond, and Ar is phenyl, Ar is not substituted by NH₂. In some embodiments, where L and A together are —CH(CH₃)₂, Ar is phenyl, and X′ is a covalent bond, R² and R′ are not both isopropyl. In other embodiments, wherein Ar is phenyl, X′ is a covalent bond, X and Q together are benzyl and L and A together are CH₂CH₂COOCl or CH₂CH₂COOH, Ar cannot be substituted by a single alkoxy, and in particular a single methoxy group.

In certain embodiments of compounds having Formula I, R⁸ and R⁹, together with the nitrogen to which they are attached, form a substituted or unsubstituted heterocyclyl. In some such embodiments, the heterocyclyl is selected from the group consisting of pyrrolidinyl, piperidinyl, morpholinyl, thiomorpholinyl and the like.

In some embodiments of compounds having Formula I, W¹ is CR¹, W² is CR², W³ is CR³, X′ is a covalent bond, and Ar is phenyl, to give compounds of Formula V:

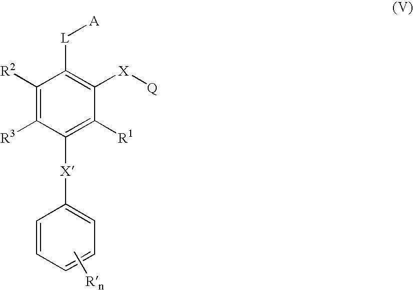

wherein A, L, X, Q, R′, R¹, R², and R³ are as defined above, and n=1-5. In some embodiments, A is hydrogen, —COOR, —C(O)NROH, —C(O)CF₃, —B(OH)₂, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR. In other embodiments, L is —(CR⁴R⁵)ₘ—, —O—(CR⁴R⁵)ₘ—, —S(O)_{q}—(CR⁴R⁵)ₘ—, —R—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—. In yet other embodiments, L is —(CR⁴R⁵)ₘ— or —O—(CR⁴R⁵)ₘ—. In some other embodiments, L and A together are —(CR⁴R⁵)ₘ—COOR or —O—(CR⁴R⁵)ₘ—COOR.

In some embodiments of compounds having Formula I, W¹ is CR¹, W² is CR², W³ is CR³, Ar is phenyl and X′ is CH₂, to give compounds of Formula VI:

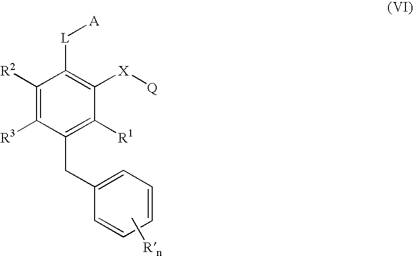

wherein A, L, X, Q, R′, R¹, R², and R³ are as defined above, and n=1-5. In some embodiments, A is hydrogen, —COOR, —C(O)NROH, —C(O)CF₃, —B(OH)₂, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR. In other embodiments, L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—. In yet other embodiments, L is —(CR⁴R⁵)ₘ— or —O—(CR⁴R⁵)ₘ—. In some embodiments, L and A together are —(CR⁴R⁵)ₘ—COOR or —O—(CR⁴R⁵)ₘ—COOR.

In another aspect, the present invention provides a pharmaceutical composition, comprising a pharmaceutically effective amount of a compound as described herein and a pharmaceutically acceptable carrier or diluent.

In accordance with yet another aspect of the present invention, there are provided methods for the inhibition of cell entry by viruses and the treatment of viral infections. In particular there are provided methods for the inhibition of cell entry by viruses, the methods comprising contacting a virus with a compound described herein. Viruses that may be inhibited by these methods include HIV, ebola, HRSV, and influenza. The methods of treatment of viral infections include administering a pharmaceutical composition of a compound described herein to a subject in need thereof. Viral infections that may be treated using this method include HIV, ebola, HRSV, and influenza infection. Typically the viral infection is HIV infection (AIDS).

In still another aspect, the present invention provides methods of preparing a compound having Formula I, wherein X′ is a covalent bond or NH, the methods comprising

reacting a compound of Formula III

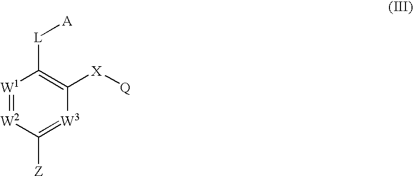

with a compound of Formula IV

Z′-Ar (IV)

in the presence of a palladium catalyst, a base, and a solvent,

under conditions suitable to form a compound of Formula I, wherein X′ is a covalent bond or NH, and wherein

A, Ar, L, X, Q, W¹, W², and W³ are as defined herein;

Z is B(OR″)₂ or NH₂, and Z′ is I, Br, Cl, or OTf; or

Z is I, Br, Cl, or OTf, and Z′ is B(OR″)₂ or NH₂; and

wherein each R″ is independently hydrogen or substituted or unsubstituted alkyl, or, each R″, together with B and the atoms to which they are attached, form a cyclic boronate.

Conditions to perform the Pd-catalyzed cross coupling reaction between organoboron compounds and organic halides or triflates, known as the Suzuki reaction, are well known in the art [“Recent Advances in the Cross-Coupling Reactions of organoboron Derivatives with Organic Electrophiles”, A. Suzuki J. Organomet. Chem. 1999, 576, 147-168]. Typically, the palladium catalysts contemplated for use in the practice of the present invention include Pd₂(dba)₃, Pd(OAc)₂, PdCl₂(PPh₃)₂ and Pd(PPh₃)₄, among others. Suitable bases contemplated for use in the practice of the present invention include inorganic bases, such as Na₂CO₃, K₂CO₃, NaOtBu, and K₃PO₄, and organic bases, such as TEA, DIEA, DIA and DBU, while suitable solvents include DMF, toluene, or a mixture of DME, ethanol and toluene.

The invention further provides methods of preparing compounds of Formula I wherein X′ is O, the methods comprising

reacting a compound of Formula III

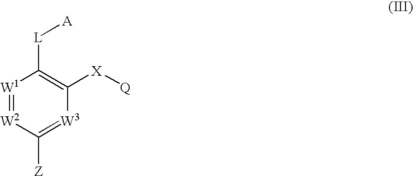

with a compound of Formula IV

Z′-Ar (IV)

in the presence of a copper catalyst, a base, and a solvent,

under conditions suitable to form a compound of Formula I in which X′ is O, wherein

A, Ar, L, X, Q, Z, W¹, W², and W³ are as defined herein;

Z is OH, and Z′ is I, Br, Cl, or OTf; or Z is I, Br, Cl, or OTf, and Z′ is OH.

Conditions to perform the Cu-catalyzed cross coupling reaction between phenol compounds and organic halides are well known in the art [“Recent Advances in Diaryl Ether Synthesis”, J. S. Sawyer Tetrahedron 2000, 56, 5045-5065]. Typically, the copper catalysts contemplated for use in the practice of the present invention include a Cu(I) catalyst such as CuI, CuBrSMe₂, Cu(OAc)₂, CuCl, and (CuOTf)₂.PhH. Suitable bases contemplated for use in the practice of the present invention include inorganic bases, such as Cs₂CO₃ and K₂CO₃, and organic bases, such as TEA, and suitable solvents include acetonitrile, toluene, benzene and the like. The reaction is preferably conducted in the presence of a solubilizing entity, e.g., an organic ester such as EtOAc.

In still another aspect, the present invention provides methods of preparing a compound having Formula I, wherein X′ is —CH(OH)—, the methods comprising

reacting a compound of Formula III

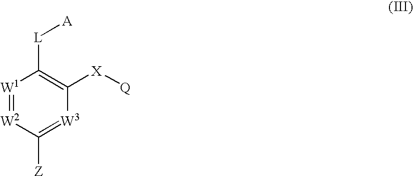

with a compound of Formula IV

Z′-Ar (IV)

in the presence of a solvent,

under conditions suitable to form a compound of Formula I wherein X′ is —CH(OH)—, and wherein

A, Ar, L, X, Q, W¹, W², and W³ are as defined herein;

Z is Li, and Z′ is —C(O)—H; or Z is —C(O)—H, and Z′ is Li.

Suitable solvents contemplated for use in the practice of the present invention include ethereal solvents such as THF and diethylether.

The compound of Formula I wherein X′ is —CH₂— can be obtained, for example, by treating the compound of Formula I wherein X′ is —CH(OH)— with a reducing agent in a solvent. Reducing agents contemplated for use in the practice of the present invention include H₂ in the presence of Pd/C or triethylsilane with trifluoroacetic acid. Suitable solvents include EtOAc and DCM.

In still another aspect, the invention provides intermediates for use in the synthesis of compounds of Formula I, the intermediates having the Formula III:

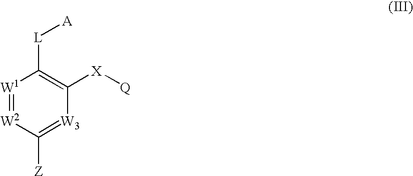

wherein,

A is hydrogen, OH, NO₂, —COOR, —C(O)NROH, —C(O)CF₃, —B(OH)₂, —SO₃H, —PO₃R₂, —OPO₃R₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR;

L is —(CR⁴R⁵)ₘ—, —O—(CR⁴R⁵)ₘ—, —S(O)_{q}—(CR⁴R⁵)ₘ—, —NR—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, —NR—C(O)NR—(CR⁴R⁵)ₘ—, —S(O)₂—NR—(CR⁴R⁵)ₘ—, or —NR—S(O)₂—(CR⁴R⁵)ₘ—;

W¹ is N or CR¹;

W² is N or CR²;

W³ is N or CR³;

X is —(CR⁶R⁷)ᵣ—, —O—(CR⁶R⁷)ᵣ—, —S(O)_{q}—(CR⁶R⁷)ᵣ—, —NR—(CR⁶R⁷)ᵣ—, —C(O)O—(CR⁶R⁷)ᵣ—, —(O)NR—(CR⁶R⁷)ᵣ—, —NR—C(O)—O(CR⁶R⁷)ᵣ—, —NR—C(O)NR—(CR⁶R⁷)ᵣ—, —S(O)₂—NR—(CR¹R⁷)ᵣ—, or —NR—S(O)₂—(CR⁶R⁷)ᵣ—;

Q is a substituted or unsubstituted cycloalkyl, substituted or unsubstituted cycloalkenyl, substituted or unsubstituted aryl, substituted or unsubstituted aralkyl, substituted or unsubstituted heterocyclyl, or substituted or unsubstituted heterocyclylalkyl;

Z is B(OR″)₂, NH₂, OH, I, Br, Cl, C(O)—H, Li or OTf;

wherein each R″ is independently hydrogen or substituted or unsubstituted alkyl, or, each R″ together with B and the atoms to which they are attached, form a cyclic boronate;

R at each occurrence is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted (C₀₋₄ alkylene)(C₆₋₁₀ aryl), or substituted or unsubstituted (C₀₋₄ alkylene)(C₁₋₉ heterocyclyl);

m=0-4;

each q is independently 0-2; and

r=0-3;

and stereoisomers thereof, tautomers thereof, and solvates thereof.

### DETAILED DESCRIPTION OF THE INVENTION

In one aspect, the compounds according to the present invention generally comprise a first planar moiety directly or indirectly attached to an acidic moiety, a hydrophobic planar moiety, and a second planar moiety, as described above. In another aspect, compounds of the present invention are defined by Formulas I and II, as described above. While not wishing to be bound by theory, compounds of the invention are believed to be inhibitors of gp41 folding in viruses such as HIV. Specifically, the compounds are believed to inhibit the formation of a fusion-critical hexameric helical bundle conformation of gp41. Thus, the compounds are believed to interfere with viral entry into cells of the host organism.

Compounds of the present invention include stereoisomers as well as optical isomers, e.g. mixtures of enantiomers as well as individual enantiomers and diastereomers, which arise as a consequence of structural asymmetry in selected compounds of the present series. The present invention also includes isomers and isoforms, defined below, of the compounds of Formula I.

The term tautomers refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having Formula I are within the scope of the present invention.

The compounds of Formula I may also be solvated, especially hydrated. Hydration may occur during manufacturing of the compounds or compositions comprising the compounds, or the hydration may occur over time due to the hygroscopic nature of the compounds.

Certain compounds within the scope of Formula I are derivatives referred to as prodrugs. The expression “prodrug” denotes a derivative of a direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrug Kinetics,” Methods in Enzymology 112:309-323 (1985); Bodor, N., “Novel Approaches in Prodrug Design,” Drugs of the Future 6:165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, New York (1985), Goodman and Gilmans, The Pharmacological Basis of Therapeutics, 8th ed., McGraw-Hill, Int. Ed. 1992. The preceding references and all references listed herein are hereby incorporated in their entirety by reference.

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and omithine. Acidic amino acids include, for example, aspartic acid and glutamic acid.

Generally, reference to a certain element such as hydrogen or H is meant to include all isotopes of that element. For example, if an R group is defined to include hydrogen or H, it also includes deuterium and tritium.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl, and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃—CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above (e.g., cyclopenylmethyl, cyclohexylethyl, and the like). The phrase also includes polycyclic alkyl groups such as, but not limited to, adamantyl, norbomyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms, and more preferred such groups have from 1 to 10 carbon atoms. Even more preferred such groups, also known as unsubstituted lower alkyl groups, have from 1 to 5 carbon atoms. Most preferred unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in groups such as carbonyls, carboxyls, and esters; nitrogen in groups such as imines, oximes, hydrazones, and nitriles. Preferred substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkylene)(aryl)amine, diarylamine, heterocyclylamine, (alkylene)(heterocyclyl)amine, (aryl)(heterocyclyl)amine, or diheterocyclylamine group.

The term “alkylene” refers to saturated, divalent straight or branched chain alkyl groups typically having in the range of about 1 up to about 20 carbon atoms, and “substituted alkylene” refers to alkylene groups further bearing one or more substituents as set forth above for substituted alkyl groups with respect to unsubstituted alkyl groups.

The phrase “unsubstituted aryl” refers to aryl groups that do not contain heteroatoms. Thus the phrase includes, but is not limited to, groups such as phenyl, biphenyl, anthracenyl, naphthyl by way of example. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. A preferred unsubstituted aryl group is phenyl. Unsubstituted aryl groups may be bonded to one or more carbon atom(s), oxygen atom(s), nitrogen atom(s), and/or sulfur atom(s) in the parent compound, however.

The phrase “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, and hydroxyphenyl among others.

The phrase “unsubstituted alkenyl” refers to straight and branched chain and cyclic groups such as those described with respect to unsubstituted alkyl groups as defined above, except that at least one double bond exists between two carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others. Preferred unsubstituted alkenyl groups include straight and branched chain alkenyl groups and cycloalkenyl groups having 1 to 20 carbon atoms, and more preferred such groups have from 1 to 10 carbon atoms.

The phrase “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon. Preferred unsubstituted alkenyl groups have form 2 to 20 carbons, and more preferred such groups have from 2 to 10 carbons.

The phrase “unsubstituted alkynyl” refers to straight and branched chain groups such as those described with respect to unsubstituted alkyl groups as defined above, except that at least one triple bond exists between two carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others. Preferred unsubstituted alkynyl groups have form 2 to 20 carbons, and more preferred such groups have from 2 to 10 carbons.

The phrase “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The phrase “unsubstituted aralkyl” refers to unsubstituted alkyl groups as defined above in which a hydrogen or carbon bond of the unsubstituted alkyl group is replaced with a bond to an aryl group as defined above. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group). Thus the phrase includes, but is not limited to, groups such as benzyl, diphenylmethyl, and 1-phenylethyl (—CH(C₆H₅)(CH₃)) among others.

The phrase “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom. Examples of substituted aralkyl groups include, but are not limited to, —CH₂C(═O)(C₆H₅), and —CH₂(2-methylphenyl) among others.

The phrase “unsubstituted heterocyclyl” refers to both aromatic and nonaromatic ring compounds including monocyclic, bicyclic, and polycyclic ring compounds containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridinyl, dihydropyridinyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl,. 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g., 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as, but not limited to, benzodioxolyl (e.g. 1,3-benzodioxoyl, etc.), chromanyl, isochromanyl, coumindinyl; unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene oxide and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiophene, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

The phrase “substituted heterocyclyl” refers to an unsubstituted heterocyclyl group as defined above in which one or more of the ring members is bonded to a non-hydrogen atom such as described above with respect to substituted alkyl groups and substituted aryl groups. Examples include, but are not limited to, 2-methylbenzimidazolyl, 5-methylbenzimidazolyl, 5-chlorobenzthiazolyl, 1-methyl piperazinyl, 2-phenoxy-thiophene, and 2-chloropyridinyl among others. In addition, substituted heterocyclyl groups also include heterocyclyl groups in which the bond to the non-hydrogen atom is a bond to a carbon atom that is part of a substituted and unsubstituted aryl, substituted and unsubstituted aralkyl, or unsubstituted heterocyclyl group. Examples include but are not limited to 1-benzylpiperdinyl, 3-phenythiomorpholinyl, 3-(pyrrolidin-1-yl)-pyrrolidinyl, and 4-(piperidin-1-yl)-piperidinyl.

The phrase “unsubstituted heterocyclylalkyl” refers to unsubstituted alkyl groups as defined above in which a hydrogen or carbon bond of the unsubstituted alkyl group is replaced with a bond to a heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group. In addition, a substituted heterocyclylalkyl group also includes groups in which a carbon bond or a hydrogen bond of the alkyl part of the group is replaced by a bond to a substituted and unsubstituted aryl or substituted and unsubstituted aralkyl group. Examples include but are not limited to phenyl-(piperidin-1-yl)-methyl and phenyl-(morpholin-4-yl)-methyl.

The phrase “unsubstituted heteroaryl” refers to unsubstituted heterocyclyl groups which are aromatic. The phrase “substituted heteroaryl” has the same meaning with respect to unsubstituted heteroaryl groups as substituted heterocyclyl groups have with respect to unsubstituted heterocyclyl groups. Examples of substituted and unsubstituted heteroaryl groups are given above under substituted and unsubstituted heterocyclyl groups.

The phrase “unsubstituted alkoxy” refers to a hydroxyl group (—OH) in which the bond to the hydrogen atom is replaced by a bond to a carbon atom of an otherwise unsubstituted alkyl group as defined above.

The phrase “substituted alkoxy” refers to a hydroxyl group (—OH) in which the bond to the hydrogen atom is replaced by a bond to a carbon atom of an otherwise substituted alkyl group as defined above.

As employed herein, the term “biaryl” refers to any molecule having two or more aryl groups.

For medicinal use, the pharmaceutically acceptable acid addition salts, i.e., those salts in which the anion does not contribute significantly to toxicity or pharmacological activity of the organic cation, are preferred. The acid addition salts are obtained either-by reaction of an organic base of Formula I with an organic or inorganic acid, preferably by contact in solution, or by any of the standard methods detailed in the literature available to any practitioner skilled in the art. Examples of useful organic acids are carboxylic acids such as maleic acid, acetic acid, lactic acid, trifluoroacetic acid, tartaric acid, propionic acid, fumaric acid, isethionic acid, succinic acid, cyclamic acid, pivalic acid and the like; useful inorganic acids are hydrohalide acids such as HCl, HBr, HI; sulfuric acid; phosphoric acid and the like. Preferred acids for forming acid addition salts include HCl, trifluoroacetic acid, and acetic acid.

The compounds of the present invention are useful for diagnosing and treating HIV infection. Methods of administering and doses for the compounds of the present invention are addressed below. The compounds of the present invention in isotopically labelled form are useful as a diagnostic agent. The isotopically labelled form of the compound is administered to a patient and detection devices are used to create an image based on the presence of the compound in particular parts of the body. Typically, radiation imaging cameras employ a conversion medium (wherein the high energy gamma ray is absorbed, displacing an electron which emits a photon upon its return to the orbital state), photoelectric detectors arranged in a spatial detection chamber (to determine the position of the emitted photons), and circuitry to analyze the photons detected in the chamber and produce an image.

Also within the scope of the invention is the use of any of the compounds according to Formula I above, for the manufacture of a medicament for the treatment of any of the conditions discussed above.

A further aspect of the invention is a method for the treatment of a subject suffering from any of the conditions discussed above, whereby an effective amount of a compound according to Formula I above, is administered to a patient in need of such treatment. Further discussion of methods for treating patients, including doses and methods of administration is discussed below, in relation to pharmaceutical compositions.

Any compound of Formula I can be used to treat and/or diagnose any of the conditions listed above. The foregoing examples are for illustration and are not meant to limit the invention in any way. It is to be understood from these examples that the compounds of Formula (1) can be used to treat any of the foregoing conditions.

### Methods of Preparation

In one aspect, the invention provides methods for preparing the compounds of the invention, as described above. For example, the invention provides methods for the preparation of compounds of Formula I, wherein X′ is a covalent bond, by Pd catalyzed cross-coupling (Suzuki Coupling) as the key step to form the biaryl structures. Schemes 1 and 2 illustrate typical coupling procedures wherein L, A, X, Q, R², R³, R⁴, W¹, W², and W³ are as defined in Formula I. In Scheme 1, Z is a leaving group such as Cl, Br, I, or triflate (OTf) and each R″ is independently hydrogen or substituted or unsubstituted alkyl, or, taken together, a cyclic boronate.

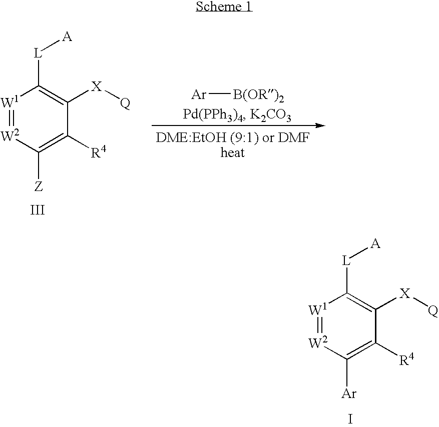

In Scheme 2, Z′ is a leaving group such as Cl, Br, I, or OTf, and R″ is as defined in Scheme 1. Those of skill in the art will readily understand that other palladium catalysts in addition to Pd(PPh₃)₄ may be used in the coupling reaction such as Pd(OAc)₂, PdCl₂(PPh₃)₂, and Pd₂(dba)₃. Similarly, in addition to K₂CO₃, other bases such as Na₂CO₃, Cs₂CO₃ or Et₃N may be employed in the coupling reaction. Suitable solvents for the reaction include DMF, DME/toluene/EtOH (9:1:1), toluene, DME, benzene, benzene/EtOH (9:1) as well as DME/EtOH (9:1).

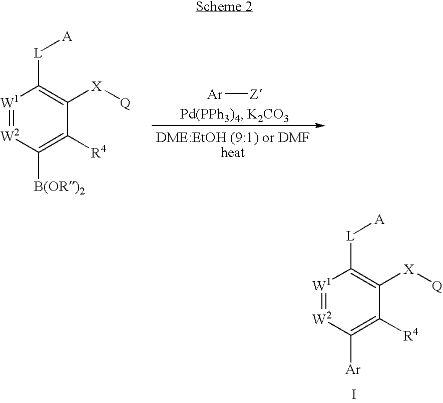

In addition, the invention provides methods for the preparation of compounds of Formula I, wherein X′ is, e.g., N or O. Scheme 3 illustrates the palladium catalyzed addition of amines to aryl and heteroaryl rings having a halide such as bromine to give compounds of the invention. The reaction is carried out in the presence of a palladium catalyst such as Pd₂(dba)₃ or others as described above, a base such as NaOtBu, and a phosphine such as tri-t-butylphosphine. Typically the reaction is performed in toluene, but benzene or other solvents may also be used. The reaction may be heated to about 60-110° C.; typically 70° C. is sufficient, but higher or lower temperatures may also be used. The X′ nitrogen may be subsequently acylated, by reaction with an activated carbonyl such as an anhydride or an acid chloride in the presence of a base such as TEA. Typically the reaction is performed in dichloromethane, but other solvents may also be used. The reaction may be heated, but typically is performed at room temperature.

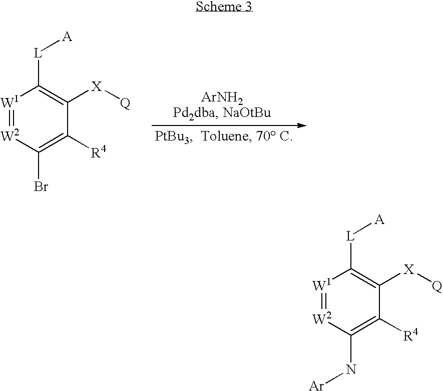

Scheme 4 shows the copper catalyzed addition of phenols or other hydroxyl containing rings to aryl and heteroaryl rings having a halide such as bromine. The reaction is carried out in the presence of about 5 mole percent EtOAc, a base such as Cs₂CO₃ and a Cu(I) catalyst such as Cul. Those of skill in the art will understand that any suitable solvent may be used; toluene works well. Typically, the reaction is heated to about 80-140° C., preferably to 110° C., but higher or lower temperatures may be used.

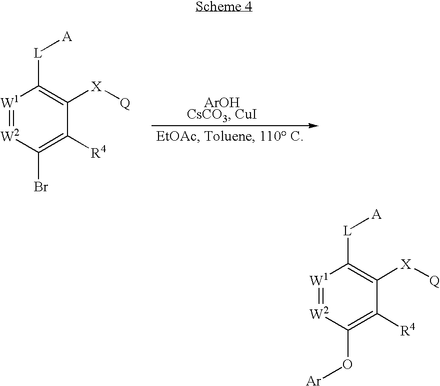

In addition, the invention provides methods for the preparation of compounds of Formula I, wherein X′ is —CH(OH)—. Scheme 5 illustrates the metal halogen exchange of aryl and heteroaryl rings having a halide such as bromine, followed by addition of aryl or heteroaryl aldehydes to the lithiated intermediate to give compounds of the invention. Typically the metal halogen exchange reaction is performed by reaction of the halide containing compound with nBuLi in THF, but diethylether or other solvents may also be used. The lithiated species is subsequently reacted in situ with aryl or heteroaryl aldehydes. The one-pot reaction is typically performed at −78° C., but higher or lower temperatures may also be used.

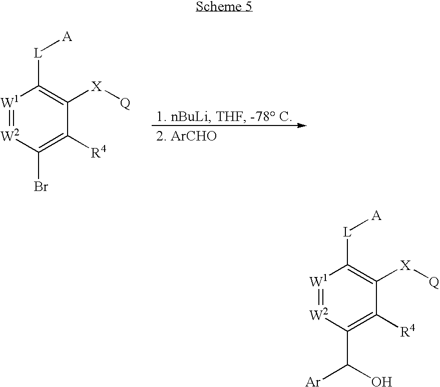

The invention also provides methods to obtain compounds of Formula I wherein X′0 is —CH₂—. Scheme 6 illustrates the reduction of compounds of Formula I wherein X′ is —CH(OH)— with a reducing agent to obtain compounds of the present invention wherin X′ is —CH₂—. Typically the reducing agent is H₂ in the presence of Pd/C or triethylsilane with trifluoroacetic acid. Typically the solvent is EtOAc or DCM, but other solvents may be used.

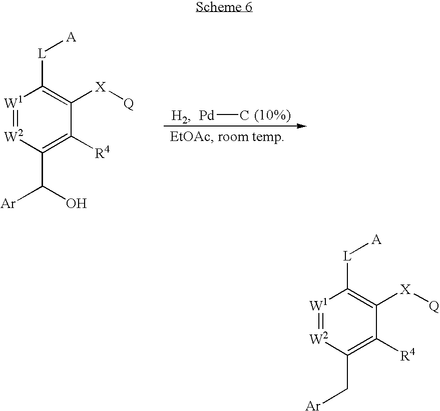

Additional techniques to make the compounds of the present invention, such as catalyzed and uncatalyzed ring closure and ring addition reactions, are well known to those of skill in the art.

### Pharmaceutical Compositions

The compounds of the present invention may be formulated as pharmaceutical compositions comprising the molecules of the present invention.

The pharmaceutical compositions of the invention can be administered to any animal that can experience the beneficial effects of the compounds of the invention. Preferably, the animal is a mammal, and foremost among such mammals are humans, although the invention is not intended to be so limited. The term “subject” as used herein therefore means any animal that can experience the beneficial effects of the compounds of the invention.

The pharmaceutical compositions of the present invention can be administered by any means that achieve their intended purpose. For example, administration can be by parenteral, subcutaneous, intravenous, intramuscular, intraperitoneal, transdermal, buccal, topical, intranasal, intrathoracic, epidural, intrathecal, intracerebroventricular or ocular routes, or by injection into the joints. Alternatively, or concurrently, administration can be by the oral route. Preferred routes of administration are oral, intravenous or intramuscular.

In addition to the pharmacologically active compounds, the new pharmaceutical preparations can contain suitable pharmaceutically acceptable carriers comprising excipients and auxiliaries that facilitate processing of the active compounds into preparations that can be used pharmaceutically.

The pharmaceutical preparations of the present invention are manufactured in a manner that is, itself, known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as carbohydrates or saccharides, for example, lactose, sucrose, dextrans, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone, and antioxidants, such as ascorbic acid or glutathione, chelating agents, low molecular weight proteins or excipients or excipients or other stabilizers and/or buffers. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as, magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses. Detergents can also be used to stabilize or to increase or decrease the absorption of the pharmaceutical composition, including liposomal carriers.

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the compound of the invention and on its particular physio-chemical characteristics.

Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of compound in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., compound or compounds of the present invention). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions of the invention, when administered orally, can be protected from digestion. This can be accomplished either by complexing with additional components in a composition to render it resistant to acidic and enzymatic hydrolysis or by packaging the compound(s) of the present invention in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical, transdermal administration, the agents are formulated into ointments, creams, salves, powders and gels. Transdermal delivery systems can also include, e.g., patches.

Compositions of the invention can also be administered in sustained delivery or sustained release mechanisms, which can deliver the formulation internally. For example, biodegradable microspheres or capsules or other biodegradable polymer configurations capable of sustained delivery of a compound can be included in the formulations of the invention (see, e.g., Putney (1998) Nat. Biotechnol. 16:153-157).

For inhalation, the compounds of the invention can be delivered using any system known in the art, including dry powder aerosols, liquids delivery systems, air jet nebulizers, propellant systems, and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Compositions of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are disclosed in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver the compound(s) of the invention directly to the brain. Parenteral administration is a preferred route of delivery if a high systemic dosage is needed. Actual methods for preparing parenterally administrable compositions will be known or apparent to those skilled in the art and are disclosed in detail, in e.g., Remington's,. See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising the compounds of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble compounds of the invention have been attached to the surface of the monolayer or bilayer. For example, the compounds of the invention can be attached to hydrazide-PEG-(distearoylphosphatidyl)ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the compounds of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467; U.S. Pat. Nos. 4,235,871, 4,501,728 and 4,837,028.

The pharmaceutical compositions of the invention can be administered in a variety of unit dosage forms depending upon the method of administration. Dosages for pharmaceutical compositions are well known to those of skill in the art. Such dosages are typically advisorial in nature and are adjusted depending on the particular therapeutic context, patient tolerance, etc. The amount of compound adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. Any factors which are normally considered when determining the individual regimen and dosage level as the most appropriate for a particular patient can be considered when determining dosages. Preferred unit dosages are from about 1 gram to about 1 mg, about 700 mg to about 5 mg, about 650 mg to about 10 mg, about 600 mg to about 20 mg, about 550 mg to about 25 mg, about 500 mg to about 30 mg, about 450 mg to about 40 mg, about 400 mg to about 50 mg, about 350 mg to about 100 mg, about 300 mg to about 150 mg, about 350 mg to about 200 mg. Even more preferably, the dosages are from about 150 mg to about 5 mg, from about 100 mg to about 5 mg, from about 50 mg to about 5 mg, from about 25 mg to about 5 mg, from about 20 mg to about 5 mg, from about 15 to about 5 mg, from about 10 to about 5 mg, from about 10 mg to about 1 mg, from about 10 mg to about 2 mg, from about 10 mg to about 4 mg, and from about 5 to about 0.5 mg.

Other pharmaceutical preparations which can be used orally include push-fit capsules made of gelatin, as well as soft, sealed capsules made of gelatin and a plasticizer, such as, glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids, such as, fatty oils or liquid paraffin. In addition, stabilizers may be added.

Suitable formulations for parenteral administration include aqueous solutions of the active compounds in water-soluble form, for example, water-soluble salts, alkaline solutions and cyclodextrin inclusion complexes. Especially preferred salts are hydrochloride and acetate salts. One or more modified or unmodified cyclodextrins can be employed to stabilize and increase the water solubility of compounds of the present invention. Useful cyclodextrins for this purpose are disclosed in U.S. Pat. Nos. 4,727,064, 4,764,604, and 5,024,998.

In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example, sodium carboxymethyl cellulose, sorbitol, and/or dextran. Optionally, the suspension may also contain stabilizers.

Compounds of Formula I can be labelled with radioactive iodine as described below or by using an exchange reaction. Exchange of hot iodine for cold iodine is well known in the art. Alternatively, a radioiodine labelled compound can be prepared from the corresponding bromo compound via a tributylstannyl intermediate. See, U.S. Pat. No. 5,122,361, herein incorporated by reference.

The present invention also includes compositions comprising a compound of Formula I complexed with a radioactive atom.

The present invention also includes diagnostic compositions, comprising a pharmaceutically acceptable carrier and a diagnostically effective amount of compositions derived from the compounds of Formula I.

The “diagnostically effective amount” of the composition required as a dose will depend on the route of administration, the type of mammal being treated, and the physical characteristics of the specific mammal under consideration. These factors and their relationship to determining this dose are well known to skilled practitioners in the medial diagnostic arts. Also, the diagnostically effective amount and method of administration can be tailored to achieve optimal efficacy but will depend on such factors as weight, diet, concurrent medication and other factors which those skilled in the medical arts will recognize. In any regard, the dose for imaging should be sufficient for detecting the presence of the imaging agent at the site of a thrombus in question. Typically, radiologic imaging will require that the dose provided by the pharmaceutical composition position of the present invention be about 5 to 20 μci, preferably about 10 μCi. Magnetic resonance imaging will require that the dose provided be about 0.001 to 5 mmole/kg, preferably about 0.005 to 0.5 mmole/kg of a compound of Formula I complexed with paramagnetic atom. In either case, it is known in the art that the actual dose will depend on the specific application.

“Pharmaceutically acceptable carriers” for in vivo use are well known in the pharmaceutical art, and are described, for example, in Remington's Pharmaceutical Sciences, Mack Publishing Co. (A. R. Gennaro edit. 1985). The pharmaceutical compositions of the present invention may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection, or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized.

The present invention also encompasses diagnostic compositions prepared for storage or administration. These would additionally contain preservatives, stabilizers and dyes. For example, sodium benzoate, sorbic acid and esters of p-hydroxybenzoic acid may be added as preservatives. In addition, antioxidants and suspending agents may be used.

The radioactive atoms associated with the compositions and diagnostic compositions of the present invention are preferably imaged using a radiation detection means capable of detecting gamma radiation, such as a gamma camera or the like.

The following examples are illustrative, but not limiting, of the method and compositions of the present invention. Other suitable modifications and adaptations of the variety of conditions and parameters normally encountered and obvious to those skilled in the art are within the spirit and scope of the invention.

### Screening Methodologies

The compounds of the present invention can be screened against potential targets using high throughput assays and secondary screening procedures, which are both described more fully below. Following screening, a primary candidate is then used as a starting point for optimizing the drug activity by altering its chemical structure. Once a final structure has been selected, the compound will go into pre-clinical and clinical testing.

The first step in the screening procedure is high throughput screening. High throughput screening typically incorporates integrated robotic systems so that large numbers of test compounds can be tested for antagonist or agonist activity within a short amount of time. These methods include, but are not limited to, homogeneous assay formats such as fluorescence resonance energy transfer, fluorescence polarization, time-resolved fluorescence resonance energy transfer, scintillation proximity assays, reporter gene assays, protein fragment complementation assays, fluorescence quenched enzyme substrate, chromogenic enzyme substrate and electrochemiluminescence, as well as more traditional heterogeneous assay formats such as enzyme-linked immunosorbant assays (ELISA) or radioimmunoassays. The molecules of the present invention can be screened by procedures known in the art. Preferably, the following screening assays are used: NC-1 ELISA, HIV-1 Mediated Cell-to-Cell Fusion, and Detection of HIV-1-Mediated Cytopathic Effect (CPE) and in Vitro Cytotoxicity (Debnath et al., “Structure-Based Identification of Small Molecule Antiviral Compounds Targeted to the gp41Core Structure of the Human Immunodeficiency Virus Type I,” J. Med. Chem. 42:3203-3209 (1999)). A preferred screening assay is NC-1 ELISA. Id.

Homogeneous assays are mix-and-read style assays that are very amenable to robotic application, whereas heterogeneous assays require separation of free from bound analyte by more complex unit operations such as filtration, centrifugation or washing. These assays are utilized to detect a wide variety of specific biomolecular interactions and the inhibition thereof by small organic molecules, including protein-protein, receptor-ligand, enzyme-substrate interactions, and so on. These assay methods and techniques are well known in the art (see, e.g., High Throughput Screening: The Discovery of Bioactive Substances, John P. Devlin (ed.), Marcel Dekker, New York, 1997 ISBN: 0-8247-0067-8).

The compounds of the present invention can be screened using advanced high throughput screening techniques, such as sequential high throughput screening (SHTS) which is the iterative process of screening a sample of compounds for activity, analyzing the results, and selecting a new set of compounds for screening, based on what has been learned from the previous screens. Selection of compounds is driven by finding structure activity relationships (SARs) within the screened compounds and using those relationships to drive further selection.

Recursive partitioning (RP) is an advanced statistical methodology that can be used in conjunction with advanced high throughput screening techniques, such as SHTS, by identifying relationships between specific chemical structural features of the molecules and biological activity. The premise is that the biological activity of a compound is a consequence of its molecular structure. Accordingly, it is very useful to identify those aspects of molecular structure that are relevant to a particular biological activity. By gaining a better understanding of the mechanism by which the compound acts, additional compounds for screening can more accurately be selected.

Quantitative structure activity relationship (QSAR) models are determined using sets of compounds whose molecular structure and biological activity are known, a training set. QSAR approaches are either linear or nonlinear. The linear approach assumes that the activity varies linearly with the level of whatever features affect it, and that there are no interactions among the different features.

Nonlinear QSAR approaches account for the fact that activity can result from threshold effects; a feature must be present at least some threshold level for activity to occur. Furthermore, interactions between features are observed in many QSAR settings, the utility of one feature depending upon the presence of another. Activity may require the simultaneous presence of two features. In particular, a molecule may be active if some optimal distance separates two features. If the features are too close, the compound is inactive. If the features are too far apart, then the compound is inactive.

Recursive partitioning (RP) methods (Hawkins, D. M. and Kass, G. V., (1982) Automatic Interaction Detection. In Topics in Applied Multivariate Analysis; Hawkins, D. H., Ed.; Cambridge University Press, pp. 269-302; Breiman, L., Friedman, J. Olshen, R. and Stone, C. (1984) Classification and Regression Trees. Chapman and Hall) can be used in nonlinear QSAR analysis. RP methods are able to model nonlinear relationships, even in the presence of strong interaction between the predictors. The output of a recursive partitioning analysis is a dendrogram (tree) in which predictors are used to progressively split the data set into smaller and more homogeneous subsets. If some node in the dendrogram contains mainly active compounds, then the detailed path by which its molecules are split out provides a clue to the molecular structures that are associated with activity. The path to a node whose cases are predominantly inactive is a clue to the molecular structures that have no bearing on or that actively inhibit activity. Hawkins, D. M., Young, S. S., and Rusinko, A. III (1997) “Analysis of a large structure-activity data set using recursive partitioning,” Quant. Struct.-Act. Relat. 16:296-302 (1997) provide an illustration of the analysis of a screening data set using FIRM.

There are two standard uses for the dendrogram. First, its structure provides an indication of which predictors are important for explaining the dependent variable. The other use is as a method of prediction; by following a future case with unknown dependent variable down to the final terminal node into which it falls based upon its independent variables, one may use the mean of the data in that node as a predictor of the new observation. Rusinko, A. III, Farmen, M. W., Lambert, C. G., Brown, P. L., and Young, S. S. (1999) “Analysis of a large structure/biological activity data set using recursive partitioning,” J. Chem. Inf. Comput. Science 39:1017-1026 demonstrate the predictive power of RP to achieve a 1500% hit rate increase over random for MAO inhibitors.

Following primary screening procedures, which identify a set of molecules for further study, secondary screening procedures can be used. Secondary screening operations differ from primary screening assays mostly in their requirements for lower throughput and the need to handle more complicated functional assay protocols. Secondary screening can be as simple as a confirmatory assay subsequent to a high throughput screen or as complicated as a cellular assay involving complicated cell functions or the measurement of intracellular phenomena. Appropriate screening assays will be well known to those of skill in the art.

Preferred secondary screening assays are assays based on interactions between the compounds of the present invention and living cells, cellular structures or biomolecules. Preferred are cell-based assays which provide information about how a compound is likely to interact in a biological system, not just about how it interacts with a potential drug target. Accordingly, cell-based screening assays provide information regarding potential interactions that may occur within the cell, such interactions that may potentially impact efficacy and/or safety of the compounds being evaluated.

Information from cellular assays can include cell morphology and/or temporal or spatial information about the cell (and components of the cell)—captured by automated cell analysis systems. These systems typically use image analysis technologies to capture the data and sophisticated informatics software to analyze the results. Alternatively, cell-based assays can use luciferase reporter-gene assays to monitor the impact of a compound. Still another approach involves electrophysiological methods that measure changes in ion concentrations (often focusing on calcium ions) in a cell. An assay can also detect changes in the potential of the cell membrane. Alternatively, assays can be performed that determine the proliferative rate of a target cell population or the rate of apoptosis.

Cell-based assays can provide information relating to compound properties such as absorption, permeability, selectivity, specificity, and metabolism. As a result, more information is known about the lead compounds that are selected after cell-based screening. The advantages of using intact, living cells for compound screening include: efficacy of compounds can be best predicted by measuring biological behaviour and function in intact cells; molecular interactions can be evaluated within the context of the “working environment” inside the cell; toxicity and nonspecific effects can be identified; drug effects on selective cell types can be distinguished; drug penetration can be evaluated in whole cell studies; orphan targets require cell based functional assays; whole cell assays obviate protein purification & expression steps.

Examples of secondary cell-based assays include viral titer assays, second messenger assays like luciferase, and fluorescent signal assays. A preferred secondary assay is one based on receptor interaction and signal transduction. Secondary screening techniques are designed to capture complex cellular activities like morphology changes, differentiation, locomotion, apoptosis, adhesion, translocations of signaling molecules, protein trafficking. (Asa D., “Automating Cell Permeability Assays,” Screening 1:36-37 (2001); Giacomello, E. et al., “Centrifugal Assay for Fluorescence-based Cell Adhesion adapted to the analysis of ex vivo cells and capable of determining relative binding strength,” BioTechniques 26:758-766 (1999); Neumayer, J. and R. Perris, “Cytotoxicity Studies Using Microplate Fluorometry for Quantification,” BioForum International 2(4): 173-176 (1998); Parker G. J. et al., “Development of High Throughput Screening Assays using Fluorescence Polarization: nuclear receptor-ligand binding and kinase/phosphatase assays,” Journal of Biomolecular Screening 5(2):77-88 (2000)).

Another major application of cell-based assays is in toxicity screening. A crucial part of drug discovery and development is the screening of drug candidates to eliminate compounds that will cause side effects. Cell-based assays can offer less-expensive, higher-throughput ways to eliminate many of the compounds that may fail these more expensive assays.

From the secondary assays, one or more lead compounds is (are) discovered. The lead compound often must be optimized to improve potency (typically from 1-5 uM to 1-10 nM) against a specific molecular target, selectivity (100-fold versus related targets) and absence of cytotoxicity, and that physical and chemical properties are appropriate for good oral bioavailability. Correlative and predictive tools to can be used to optimize lead compounds toward development. These include software models that describe molecular characteristics of poorly absorbed or toxic compounds, and experimental assays, including Caco-2 permeability models and liver microsome metabolism models, to facilitate the optimization process.

Following lead optimization, an animal model is developed which in essence determines not only the compound's affect on the body, but the body's affect on the compound. For example, in animal testing, the amount of compound absorbed into the blood, how the compound is broken down chemically in the body and the toxicity of its breakdown products (i.e. metabolites) is measured. Animals can also give information regarding how quickly the compound and its metabolites are excreted from the body. A metabolite of the compound can be more effective than the compound originally picked for development. Accordingly, metabolites of the compounds of the present invention are encompassed in the present invention. Preferably, two or more species of animal are typically tested because a compound may affect one species differently from another.

Following animal testing and FDA approval, testing in humans is conducted. Phase 1 clinical trials mainly determines the safety of the drug, while Phase 2 clinical trials mainly determine the effectiveness of the drug, along with short-term safety. Phase 3 trials test safety, effectiveness and dosage. Guidelines for animal testing and human testing are known to one of skill in the art, and can be found on the Food and Drug Administration's (FDA) web page.

The following examples are for illustration and are not intended to limit the scope of the present invention in any manner.

### EXAMPLES

The following abbreviations are used throughout the application with respect to chemical terminology:

aq Aqueous

Bn Benzyl

tBu tert-Butyl

BuLi Butyl lithium

DBU 1,5-Diazabicyclo[4.3.0]non-5-ene

DCM Dichloromethane

Deg Degrees

DIA Diisopropylamine

DIEA Diisopropylethylamine

DME Dimethoxyethane

DMF N,N-Dimethylformamide

DMSO Dimethylsulfoxide

Dppf Ph₂PC₅H₄FeC₅H₄pPh₂

Eqv. Equivalents

EtOAc Ethyl acetate

EtOH Ethanol

HPLC High Pressure Liquid Chromatography

IC₅₀ value The concentration of an inhibitor that causes a 50% reduction in a measured activity.

LAH Lithium aluminum hydride

Me Methyl

MeCN Acetonitrile

MeOH Methanol

OTf Triflate, i.e. —OSO₂CF₃

Pd₂(dba)₃ Tris(dibenzylideneacetone)dipalladium(0)

Ph Phenyl

rt Room temperature

TEA Triethylamine

TES Triethylsilane

TFA Trifluoroacetic acid

THF Tetrahydrofuran

Tol Toluene

### Example 1

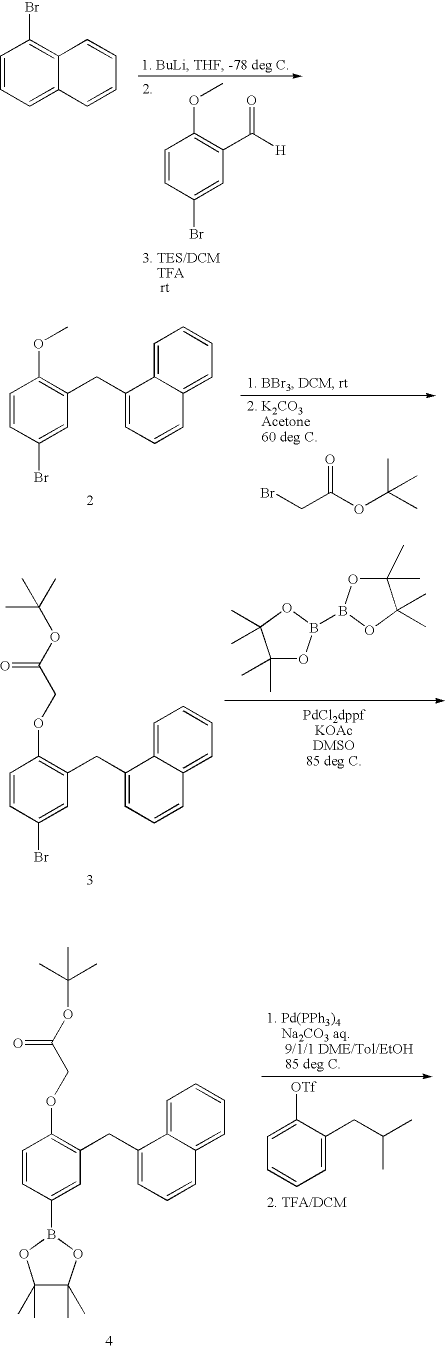

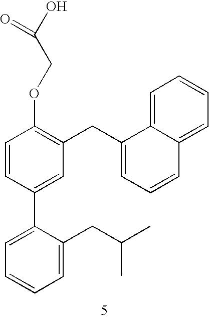

1-(5-Bromo-2-methoxy-benzyl)-naphthalene, 2. To a solution of 1-bromonaphthalene 1 (1.70 mL, 12.22 mmol) in 40 mL of THF at −78° C. was added n-BuLi (7.64 mL of a 1.6 M solution in hexanes, 12.22 mmol, 1.00 eqv) via syringe. After stirring for 1 h, a solution of 4-bromo-anisaldehyde (2.63 g, 12.23 mmol, 1.00 eqv) in 30 mL of THF was added dropwise via a dropping funnel. The resulting mixture was stirred for 60 min at −78° C., then for 2 h at −30° C. and finally 15 h at rt. The reaction was quenched with 1 M aq HCl and added to Et₂O (100 mL). The organic phase was separated, washed with H₂O, dried over MgSO₄ and concentrated in vacuo. Column chromatography (CH₂Cl₂) yielded 3.70 g of a pale greenish foam (88%): ¹H NMR (500 MHz, CDCl₃) δ 3.84 (s, 3H), 6.80 (d, J=8.51 Hz, 1H), 6.81 (s, 1H), 7.18 (d, J=2.68 Hz, 1H), 7.34 (dd, J₁=8.82 Hz, J₂=2.52 Hz, 1H), 7.48 (m, 3H), 7.55 (d, J=7.09 Hz, 1H), 7.79 (d, J=8.20 Hz, 1H), 7.87 (m, 1H), 8.00 (m, 1H); HRMS (EI) Calcd for C₁₈H₁₅BrO₂: 342.0255. Found 342.0251.

To a solution of the compound obtained in the previous reaction (0.57 g, 1.6 mmol) in 30 mL of 1:1 DCM/triethylsilane (TES) was added 1 mL of TFA. The resulting solution was allowed to stand at rt for 5 h. The mixture was concentrated in vacuo to yield 0.52 g of a white foam (91%): %): ¹H NMR (400 MHz, CDCl₃) δ 3.87 (s, 3H), 4.37 (s, 2H), 6.79 (d, J=8.0 Hz 1H), 6.94 (s, 1H), 7.26 (m, 2H), 7.46 (m, 3H), 7.77 (d, J=8.0 Hz, 1H), 7.87 (d, J=8.0 Hz, 1H), 7.94 (d, J=8.0 Hz, 1H).

(4-Bromo-2-naphthalen-1-ylmethyl-phenoxy)-acetic acid tert-butyl ester, 3. To a solution of 2 (0.52 g, 1.6 mmol) in 100 mL of DCM was added 4.8 mL of a 1 M solution of BBr₃ (3 eqv., 4.8 mmol) in DCM. The solution was allowed to stand at rt for 5 h. The mixture was quenched with H₂O and extracted with DCM. The organic fractions were combined, dried (MgSO₄), filtered, and concentrated in vacuo to yield a crude oil. Column chromatography (30% EtOAc in Hexanes) yielded 0.42 g of a clear oil (84%): ¹H NMR (400 MHz, CDCl₃) δ 4.42 (s, 2H), 4.90 (s, 1H), 6.74 (d, J=8.0 Hz 1H), 7.12 (s, 1H), 7.26 (m, 2H), 7.44 (t, J=8.0 Hz, 1H), 7.52 (m, 2H), 7.81 (d, J=8.0 Hz, 1H), 7.91 (d, J=8.0 Hz, 1H), 8.02 (d, J=8.0 Hz, 1H).

To a mixture of the compound obtained in the previous reaction (6.78 g, 21.7 mmol) and K₂CO₃ (3 eqv., 9.0 g, 0.065 mol) in 100 mL of acetone was added tert-butyl-bromoacetate (1.05 eqv., 3.4 mL, 22.8 mmol). The solution was refluxed for 12 h. The solution was allowed to cool to rt, added to H₂O, and extracted with DCM. The organic fractions were combined, dried (MgSO₄), filtered, and concentrated in vacuo to yield a crude oil. Column chromatography (30% EtOAc in Hexanes) yielded 8.9 g of a clear oil (96%): ¹H NMR (400 MHz, CDCl₃) δ 1.51 (s, 9H), 4.48 (s, 2H), 4.60 (s, 2H), 6.67 (d, J=8.0 Hz, 1H), 6.97 (s, 1H), 7.30 (m, 2H), 7.48 (m, 3H), 7.79 (d, J=8.0 Hz, 1H), 7.89 (d, J=8.0 Hz, 1H), 8.00 (d, J=8.0 Hz, 1H).

[2-Naphthalen-1-ylmethyl-4-(4,4,5,5-tetramethyl-[1,3,2]dioxaborolan-2-yl)-phenoxy]-acetic acid tert-butyl ester,4. In a 40 mL vial were placed 3 (1.0 g, 2.34 mmol), potassium acetate (970 mg, 7.02 mmol), bis(pinacolato)diboron (654 mg, 2.57 mmol), DMSO (14 mL) and [1,1′-bis(diphenylphosphino)ferrocene]dichloropalladium (II) (86 mg, 0.12 mmol). The vial was flushed with nitrogen and capped tightly. The suspension was stirred for 3 h at 85° C. after which time the reaction was partitioned between H₂O and DCM. The aqueous layer was extracted twice with DCM. The organic fractions were combined, dried (MgSO₄), filtered, and concentrated in vacuo to yield a crude oil. Column chromatography (30% EtOAc in Hexanes) yielded 0.83 g of a clear oil (75%): ₁H NMR (400 MHz, CDCl₃) δ 1.28 (s, 9H). 1.43 (s, 6H), 1.47 (s, 6H), 4.49 (s, 2H), 4.54 (s, 2H), 6.76 (d, J=8.33 Hz, 1H), 7.16 (d, J=7.1 Hz, 1H), 7.35 (d, J=7.1 Hz, 1H), 7.45 (d, J=6.8Hz, 1H), 7.48 (d, J=6.8Hz, 1H), 7.58 (d, J=1.2 Hz, 1H), 7.67 (d, J=8.3 Hz, 1H), 7.70 (d, J=8.3 Hz, 1H), 7.84 (d, J=7.3 Hz, 1H), 8.18 (d, J=7.8 Hz, 1H).

(2′-Isobutyl-3-naphthalen-1-ylmethyl-biphenyl-4-yloxy)-acetic acid, 5. 2-Isobutylphenyl trifluoromethanesulfonate (0.100 g, 0.35 mmol), 4 (1.0 eqv., 0.17 g, 0.35 mmol) and Pd(PPh₃)₄ (10 mol %, 40 mg) were dissolved in 3 mL of 9/1/1 DME/EtOH/Toluene. Na₂CO₃ (0.35 mL of 2 M aq solution, 0.7 mmol, 2 eqv.) was added via syringe and the solution was stirred at 85° C. for 17 h. The reaction mixture was concentrated in vacuo and taken up in 2:1 H₂O/CH₂Cl₂. The layers were separated and the H₂O layer was extracted further with CH₂Cl₂. The combined organic fractions were dried (MgSO₄), filtered, and concentrated in vacuo. Column chromatography (30% EtOAc in Hexanes) yielded 0.073 g of a clear oil. The material was dissolved in 2 mL of 1:1:0.05 TFA/DCM/H₂O and allowed to stand at rt for 12 h. The solution was concentrated in vacuo to yield 0.051 g of a white solid (34%). ¹H NMR (400 MHz, CDCl₃) δ 0.57 (d, J=8.0 Hz, 6H), 1.48 (m, 1H), 2.25 (d, J=8.0 Hz, 2H), 4.57 (s, 2H) 4.82 (s, 2H), 6.88 (s, 1H), 6.90 (s, 1H), 7.14 (m, 5H), 7.31 (d, J=8.0 Hz, 1H), 7.41 (t, J=8.0 Hz, 1H), 7.46 (m, 2H), 7.75 (d, J=8.0 Hz, 1H), 7.86 (d, J=8.0 Hz, 1H), 8.06 (d, J=8.0 Hz, 1H).

### Example 2

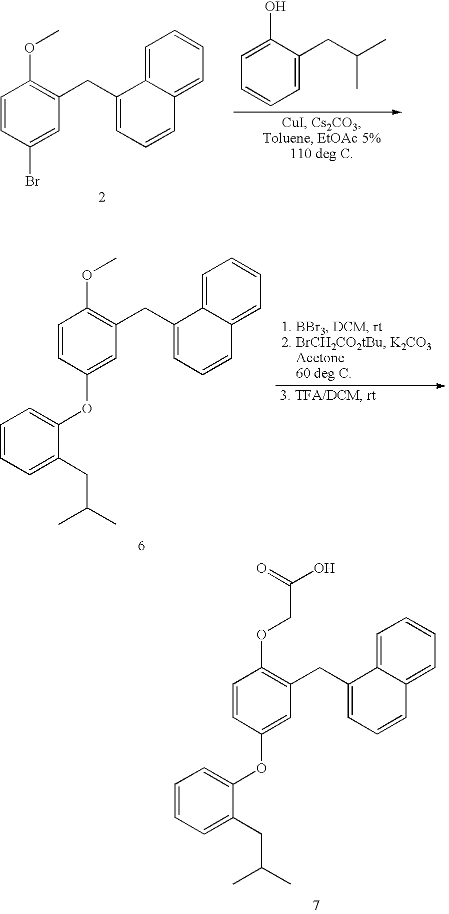

4-(2-Isobutyl-phenoxy)-1-methoxy-2-[5-methylene-6-prop-2-en-(Z)-ylidene-cyclohexa-1,3-dienylmethyl]-benzene, 6. The aryl halide 2 (40 mg, 0.12 mmol), 2-isobutylphenol (25 mg, 0.17 mmol), Cs₂CO₃ (58 mg, 0.018 mmol), Cul (1.1 mg, 0.006 mmol, 5.0 mol % Cu), ethyl acetate (0.5 mg, 0.006 mmol, 5.0 mol %) and toluene (1.0 mL) were added to a 7 mL vial which was then sealed purged with nitrogen and heated at 110° C. for 24 hours. The reaction mixture was then allowed to cool down to room temperature, diluted with Et₂O and washed sequentially with 5% aqueous NaOH, H₂O and brine. The organic layer was dried over MgSO₄ and concentrated under vacuum to give the crude product. Purification by flash chromatography (20% EtOAc in hexanes) on silica gel afforded 40 mg (85%) of the analytically pure compound. ¹H NMR (400 MHz, CDCl₃) δ 0.84 (d, J=6.5 Hz, 6H), 1.89 (m, 1H), 2.45 (d, J=7.3 Hz, 2H), 3.89 (s, 3H), 4.43 (s, 2H), 6.60 (d, J=2.8 Hz, 1H), 6.67 (d, J=8.0 Hz, 1H), 6.76 (dd, J=8.0, 3.0 Hz, 1H), 6.88 (d, J=8.0 Hz, 1H), 6.95 (dd, J=8.0, 8.0 Hz, 1H), 7.03 (dd, J=8.0, 8.0 Hz, 1H), 7.12 (d, J=8.0 Hz, 1H), 7.26 (d, J=8.0 Hz, 1H), 7.41 (dd, J=8.0, 8.0 Hz, 1H), 7.47-7.50 (m, 2H), 7.76 (d, J=8.0 Hz, 1H), 7.86-7.92 (m, 1H), 8.00-8.03 (m, 1H).

[4-(2-Isobutyl-phenoxy)-2-naphthalen-1-ylmethyl-phenoxy]-acetic acid, 7. To a solution of compound 6 (30 mg, 0.07 mmol) in 5 mL of DCM was added 0.21 mL of a 1 M solution of BBr₃ (3 eqv., 0.21 mmol) in DCM. The solution was allowed to stand at rt for 1 h. The mixture was quenched with H₂O and extracted with DCM. The organic fractions were combined, dried (MgSO₄), filtered, and concentrated in vacuum to yield a crude oil. Column chromatography (30% EtOAc in hexanes) yielded 23 mg of a clear oil (95%). ¹H NMR (400 MHz, CDCl₃) δ 0.88 (d, J=6.6 Hz, 6H), 1.93 (m, 1H), 2.49 (d, J=7.1 Hz, 2H), 4.43 (s, 2H), 6.72 (m, 3H), 6.81 (d, J=8.6 Hz, 1H), 6.98 (dd, J=7.3, 7.6 Hz, 1H), 7.28 (d, J=7.1 Hz, 1H), 7.42 (dd, J=7.1, 8.3 Hz, 1H), 7.72 (m, 2H), 7.78 (d, J=8.0 Hz, 2H), 7.89 (m, 2H), 8.07 (m, 2H).

To a mixture of the compound obtained in the previous reaction (30 mg, 0.06 mmol) and K₂CO₃ (3 eqv., 25 mg, 0.18 mmol) in 1 mL of acetone was added tert-butyl-bromoacetate (1.5 eqv., 13 μL, 0.09 mmol). The solution was refluxed for 12 h. The solution was allowed to cool to rt, added to H₂O, and extracted with DCM. The organic fractions were combined, dried (MgSO₄), filtered, and concentrated in vacuum to yield a crude oil. Column chromatography (20% EtOAc in hexanes) yielded 23 mg of a clear oil (96%). ¹H NMR (400 MHz, CDCl₃) δ 0.81 (d, J=6.5 Hz, 6H), 1.51 (s, 9H), 1.85 (m, 1H), 2.41 (d, J=7.3 Hz, 2H), 4.50 (s, 2H), 4.57 (s, 2H), 6.58 (d, J=2.5 Hz, 1H), 6.65 (d, J=8.3 Hz, 1H), 6.70 (dd, J=9.3, 2.5 Hz, 1H), 6.74 (d, J=8.0 Hz, 1H), 6.95 (dd, J=8.0, 7.8 Hz, 1H), 7.00 (dd, J=8.0, 8.1 Hz, 1H), 7.11 (d, J=8.0 Hz, 1H), 7.30 (d, J=8.0 Hz, 1H), 7.40 (dd, J=8.3, 7.8 Hz, 1H), 7.47 (m, 2H), 7.75 (d, J=8.3 Hz, 1H), 7.88 (m, 1H), 8.04 (m, 1H).

The tert-Butyl ester obtained in the previous step (20 mg, 0.40 mmol) was dissolved in 0.5 mL of 1:1 TFA/DCM and allowed to stand at room temperature for 1 h. The solution was concentrated in vacuum to yield 18 mg of a white solid (95%). ¹H NMR (400 MHz, CDCl₃) δ 0.83 (d, J=6.5 Hz, 6H), 1.87 (m, 1H), 2.43 (d, J=7.3 Hz, 2H), 4.49 (s, 2H), 4.69 (s, 2H), 6.66 (d, J=8.0 Hz, 1H), 6.70 (dd, J=8.0, 1.2 Hz, 1H), 6.74 (dd, J=8.0, 3.0 Hz, 1H), 6.80 (d, J=8.0 Hz, 1H), 6.98 (dd, J=8.0, 7.8 Hz, 1H), 7.00 (dd, J=8.0, 8.1 Hz, 1H), 7.13 (dd, J=8.0, 1.7 Hz, 1H), 7.25 (dd, J=8.0, 1.0 Hz, 1H), 7.40 (dd, J=8.0, 8.0 Hz, 1H), 7.49 (m, 2H), 7.76 (d, J=8.0 Hz, 1H), 7.88 (m, 1H), 8.02 (m, 1H).

### Example 3

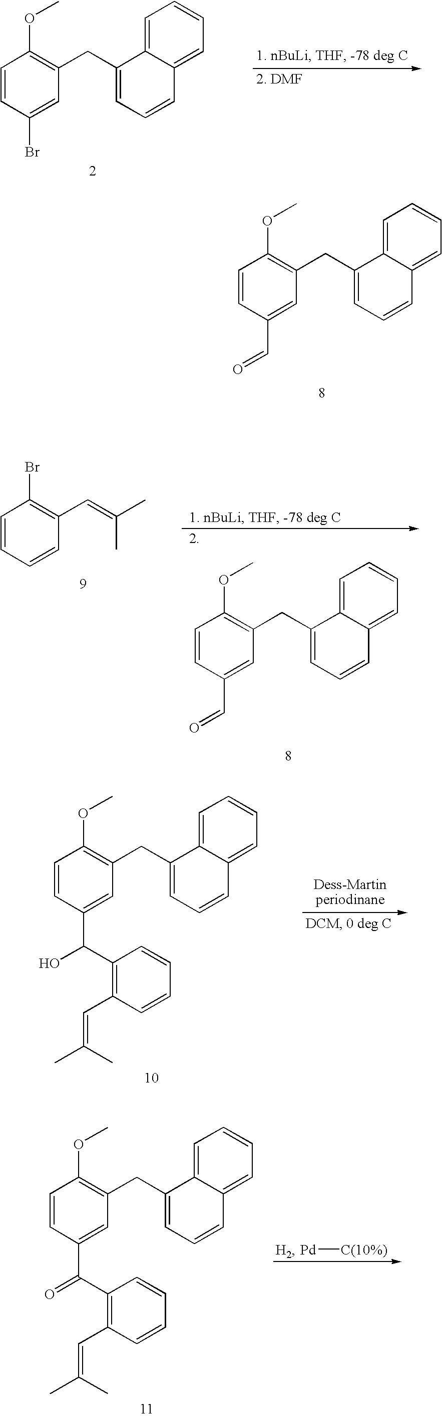

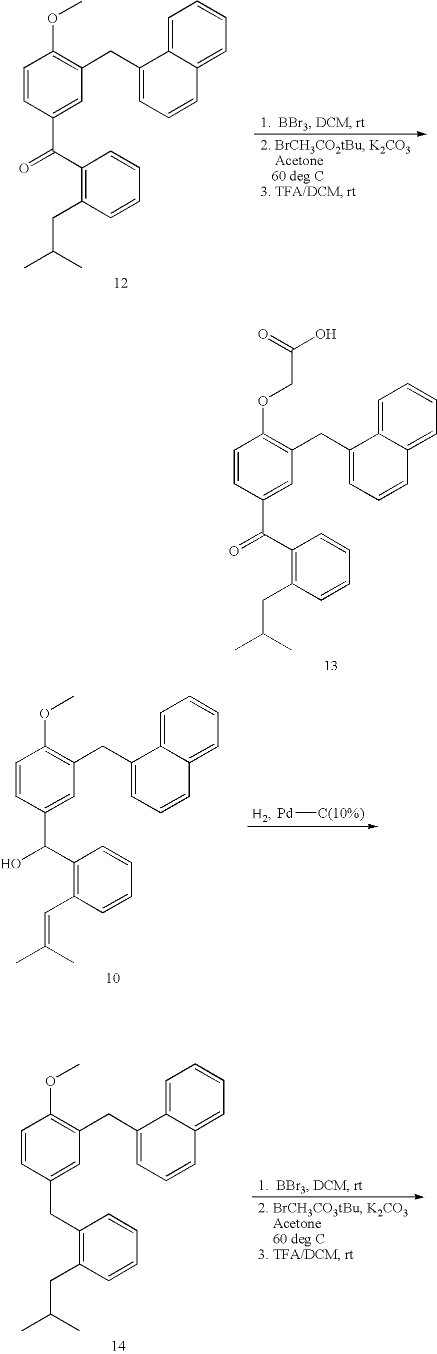

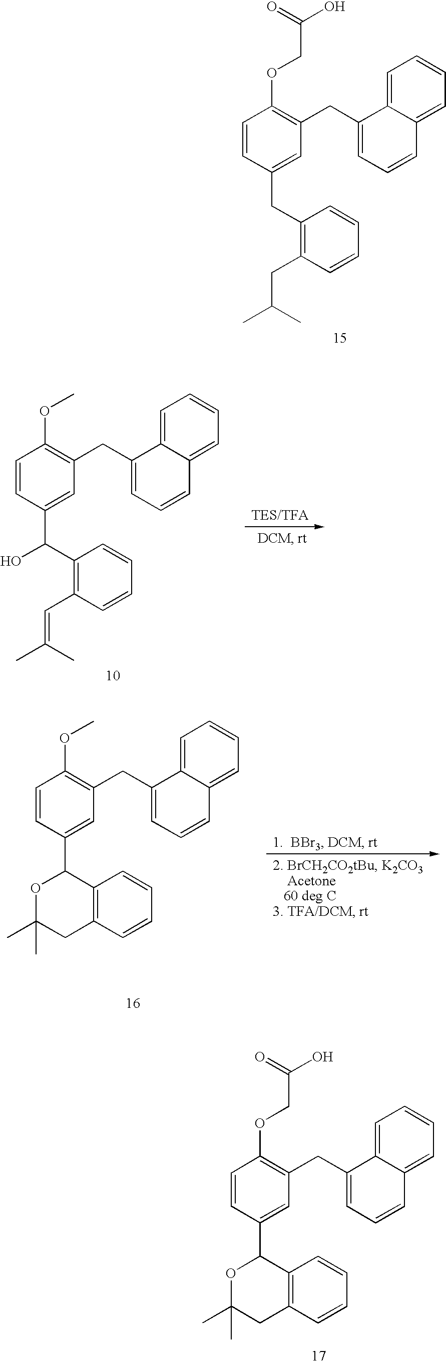

4-Methoxy-3-naphthalen-1-ylmethyl-benzaldehyde, 8. To a solution of 2 (0.5 g, 1.5 mmol) in 5 mL of THF at −78° C. was added n-BuLi (0.75 mL of a 2.0 M solution in hexanes, 1.5 mmol, 1.0 eqv.) via syringe. After stirring for 30 minutes, DMF (0.58 mL, 7.5 mmol) was added. The resulting mixture was stirred for 30 min at −78° C. The reaction was quenched with 1 M aq HCl and added to Et₂O (20 mL). The organic phase was separated, washed with H₂O, dried over MgSO₄ and concentrated in vacuum. Column chromatography (EtOAc) yielded 0.4 g of a colorless foam (95%). ¹H NMR (400 MHz, CDCl₃) δ 3.98 (s, 3H), 4.44 (s, 2H), 7.04 (d, J=8.5 Hz, 1H), 7.25 (d, J=8.5 Hz, 1H), 7.43 (m, 4H), 7.78 (m, 2H), 7.87 (m, 1H), 7.94 (m, 1H), 8.32 (m, 1H).

(4-Methoxy-3-naphthalen-1-ylmethyl-phenyl)-[2-(2-methyl-propenyl)-phenyl]-methanol, 10. To a solution of 9 (0.15, 0.72 mmol) in 5 mL of THF at −78° C. was added n-BuLi (0.36 mL of a 2.0 M solution in hexanes, 0.72 mmol, 1.00 eqv.) via syringe. After stirring for 1 h, a solution of 8 (0.20 g, 0.72 mmol, 1.00 eqv.) in 1 mL of THF was added dropwise via syringe. The resulting mixture was stirred for 30 min at −78° C. The reaction was quenched with 1 M aq HCl and added to Et₂O (10 mL). The organic phase was separated, washed with H₂O, dried over MgSO₄ and concentrated in vacuum. Column chromatography (15% of EtOAc in hexanes) yielded 0.26 g of a colorless oil.

(4-Methoxy-3-naphthalen-1-ylmethyl-phenyl)-[2-(2-methyl-propenyl)-phenyl]-methanone, 11. To a solution of 10 (30 mg, 0.07 mmol) in DCM (1 mL), Dess-Martin periodinane (46 mg, 0.11 mmol, 1.5 eqv.) was added while cooling at 0° C. After 30 min, reaction was completed, the mixture was quenched with H₂O and extracted with DCM. The organic fractions were combined, dried (MgSO₄), filtered, and concentrated in vacuum to yield a crude oil. Column chromatography (10% EtOAc in Hexanes) yielded 21 mg of a clear oil (72%). ¹H NMR (400 MHz, CDCl₃) δ 1.53 (3H, s), 1.60 (3H, s), 3.94 (s, 3H). 4.41 (s, 2H), 5.96 (s,1H), 6.93 (d, J=8.6 Hz 1H), 7.18 (m, 4H), 7.35 (m, 3H), 7.48 (m, 2H), 7.68 (dd, J=8.3, 2.3 Hz, 1H), 7.73 (d, J=7.8 Hz, 1H), 7.87 (m, 1H), 7.99 (m, 1H).

(2-Isobutyl-phenyl)-(4-methoxy-3-naphthalen-1-ylmethyl-phenyl)-methanone, 12. A mixture of 10 (20 mg, 0.045 mmol) and palladium on charcoal (10%) (0.10 mol %, 0.0045 mmol) in EtOAc (0.5 mL) were stirred under hydrogen atmosphere for 30 minutes. Then the reaction was filtered through celite and evaporated in vacuum to give 17 mg of a crude oil that was used in the next step without purification.

[4-(2-Isobutyl-benzoyl)-2-naphthalen-1-ylmethyl-phenoxy]-acetic acid, 13. To a solution of 12 (17 mg, 0.04 mmol) in 1 mL of DCM was added 0.16 mL of a 1 M solution of BBr₃ (3 eqv., 0.16 mmol) in DCM. The solution was allowed to stand at rt for 1 h. The mixture was quenched with H₂O and extracted with DCM. The organic fractions were combined, dried (MgSO₄), filtered, and concentrated in vacuum to yield a crude oil that was used in the next step without purification

To a mixture of the compound obtained in the previous step (15 mg, 0.04 mmol) and K₂CO₃ (3 eqv., 15 mg, 0.12 mmol) in 1 mL of acetone was added tert-butyl-bromoacetate (1.05 eqv., 6 μl, 0.042 mmol). The solution was refluxed for 12 h. The solution was allowed to cool to rt, added to H₂O, and extracted with DCM. The organic fractions were combined, dried (MgSO₄), filtered, and concentrated in vacuum to yield a crude oil.

The tert-Butyl ester (obtained in the previous reaction) was dissolved in 1 mL of 1:1 TFA/DCM and allowed to stand at room temperature for 12 h. The solution was concentrated in vacuum and the residue purified by HPLC to yield 10 mg of a white solid (95%). ¹H NMR (400 MHz, CDCl₃) δ 0.86 (d, J=6.5 Hz, 6H), 1.80 (m, 1H), 2.42 (d, J=6.5 Hz, 1H), 3.95 (s, 1H), 4.40 (2H, s), 6.98 (d, J=8.6 Hz, 1H), 7.02 (d, J=6.8 Hz, 1H), 7.25 (m, 3H), 7.35 (m, 2H), 7.44 (dd, J=8.0, 7.6 Hz, 1H), 7.53 (d, J=8.0, 7.6 Hz, 1H), 7.65 (d, J=7.6 Hz, 1H), 7.76 (d, J=7.6 Hz, 1H), 8.01 (d, J=8.3 Hz, 1H), 8.27 (d, J=7.6 Hz, 1H).

1-[5-(2-Isobutyl-benzyl)-2-methoxy-benzyl]-naphthalene, 14. A mixture of 10 (90 mg, 0.22 mmol) and palladium on charcoal (10%) (0.10 mol %, 0.022 mmol) in EtOAc (0.5 mL) were stirred under hydrogen atmosphere for 1 hour and 30 minutes. Then the reaction was filtered through celite and evaporated in vacuum to give 80 mg of a crude oil. Column chromatography (10% EtOAc in Hexanes) yielded 40 mg of a clear oil (46%). ¹H NMR (400 MHz, CDCl₃) δ 0.81 (d, J=6.8 Hz, 6H), 1.75 (s, 3H), 2.33 (d, J=7.3 Hz, 3H), 3.81 (s, 2H), 3.86 (s, 3H), 4.40 (s, 2H), 6.70 (s, 1H), 6.83 (d, J=8.3 Hz, 1H), 6.91 (d, J=6.8 Hz, 1H), 6.96 d, J=7.6 Hz, 1H), 7.02 (d, J=6.8 Hz, 1H), 7.21 (m, 3H), 7.40 (dd, J=7.3, 7.8 Hz, 1H), 7.47 (m, 2H), 7.75 (d, J=8.0 Hz, 1H), 7.87 (d, J=7.8 Hz, 1H), 8.01 (d, J=9.0 Hz, 1H).

[4-(2-Isobutyl-benzyl)-2-naphthalen-1-ylmethyl-phenoxy]-acetic acid, 15. To a solution of 14 (30 mg, 0.07 mmol) in 1 mL of DCM was added 0.21 mL of a 1 M solution of BBr₃ (3 eqv., 0.21 mmol) in DCM. The solution was allowed to stand at rt for 1 h. The mixture was quenched with H₂O and extracted with DCM. The organic fractions were combined, dried (MgSO₄), filtered, and concentrated in vacuum to yield a crude oil that was used without further purification.

To a mixture of the compound obtained in the previous step (26 mg, 0.07 mmol) and K₂CO₃ (3 eqv., 29 mg, 0.21 mol) in 1 mL of acetone was added tert-butyl-bromoacetate (1.05 eq., 31 μl, 0.21 mmol). The solution was refluxed for 12 h and then allowed to cool to room temperature, added to H₂O, and extracted with DCM. The organic fractions were combined, dried (MgSO₄), filtered, and concentrated in vacuum to yield a crude oil. Column chromatography (20% EtOAc in hexanes) yielded 32 mg of a clear oil (96%). ¹H NMR (400 MHz, CDCl₃) δ 0.79 (d, J=6.6 Hz, 6H), 1.49 (s, 9H), 1.71 (s, 1H), 2.32 (d, J=7.3 Hz, 2H), 3.79 (s, 2H), 4.49 (s, 2H), 7.08 (m, 4H), 7.27 (d, J=7.1 Hz, 1H), 7.45 (m, 3H), 7.75 (d, J=8.3 Hz, 1H), 7.87 (d, J=8.0 Hz, 1H), 8.04 (d, J=8.0 Hz, 1H).

The tert-Butyl ester obtained above was dissolved in 1 mL of 1:1 TFA/DCM and allowed to stand at room temperature for 12 h. The solution was concentrated in vacuum to yield 51 mg of a white solid (95%).¹H NMR (400 MHz, CDCl₃) δ 0.80 (d, J=6.5 Hz, 6H), 1.72 (s, 1H), 2.32 (d, J=7.3 Hz, 2H), 3.80 (s, 2H), 4.42 (s, H), 4.66 (s, 2H), 6.72 (m, 2H), 6.73 (d, J=8.1 Hz, 1H), 6.77 (d, J=8.1 Hz, 1H), 6.95 (m, 4H), 7.38 (t, J=8.3 Hz, 1H), 7.45 (m, 3H), 7.70 (d, J=8.0 Hz, 1H), 7.85 (d, J=8.0 Hz, 1H), 8.04 (d, J=8.0 Hz, 1H).

1-(4-Methoxy-3-naphthalen-1-ylmethyl-phenyl)-3,3-dimethyl-isochroman, 16. To a solution of 10 (10 mg, 0.02 mmol) in 0.5 mL of 1:1 DCM/triethylsilane (TES) was added 0.05 mL of TFA. The resulting solution was allowed to stand at rt for 5 minutes. The mixture was concentrated in vacuum to yield a crude oil. Column chromatography (20% EtOAc in hexanes) yielded 7 mg of a clear oil (87%). ¹H NMR (400 MHz, CDCl₃) δ 0.54 (s, 3H), 1.08 (s, 3H), 2.68 (c, J=4.5 Hz, 6H), 3.85 (s, 1H), 3.87 (s, 2H), 4.43 (c, J=7.0 Hz, 6H), 6.68 (d, J=2.2 Hz, 1H), 6.90 (m, 3H), 7.05 (dd, J=7.1, 7.1 Hz, 1H), 7.17 (m, 3H), 7.38 (dd, J=8.1, 7.1 Hz, 1H), 7.47 (m, 2H), 7.73 (d, J=8.3 Hz, 1H), 7.87 (m, 1H), 8.04 (m, 1H).

[4-(3,3-Dimethyl-isochroman-1-yl)-2-naphthalen-1-ylmethyl-phenoxyl-acetic acid, 17. To a solution of 16 (7 mg, 0.015 mmol) in 0.5 mL of DCM was added 0.045 mL of a 1 M solution of BBr₃ (3 eqv., 0.045 mmol) in DCM. The solution was allowed to stand at rt for 1 h. The mixture was quenched with H₂0 and extracted with DCM. The organic fractions were combined, dried (MgSO₄), filtered, and concentrated in vacuum to yield a crude oil that was used in next step without purification.

To a mixture of the compound obtained in the previous step (6 mg, 0.015 mmol) and K₂CO₃ (3 eqv., 6 mg, 0.045 mol) in 100 mL of acetone was added tert-butyl-bromoacetate (1.05 eqv., 2 μl, 0.015 mmol). The solution was refluxed for 12 h. The solution was allowed to cool to rt, added to H₂O, and extracted with DCM. The organic fractions were combined, dried (MgSO₄), filtered, and concentrated in vacuum to yield a crude oil.

The tert-Butyl ester was dissolved in 0.5 mL of 1:1 TFA/DCM and allowed to stand at room temperature for 12 h. The solution was concentrated in vacuum to yield 4 mg of a white solid (95%). ¹H NMR (400 MHz, CDCl₃) δ 0.55 (s, 3H), 1.09 (s, 3H), 2.68 (c, J=4.5 Hz, 6H), 3.85 (s, 1H), 4.48 (c, J=7.0 Hz, 6H), 4.67 (s, 2H), 6.68 (m, 2H), 6.91 (m, 2H), 7.05 (dd, J=7.0,7.1Hz, 1H), 7.17 (m, 3H), 7.41 (dd, J=8.0, 7.3Hz, 1H), 7.49 (m, 2H), 7.74 (d, J=8.3 Hz, 1H), 7.88 (d, J=8.1 Hz, 1H), 8.05 (d, J=8.0 Hz, 1H).

### Example 4

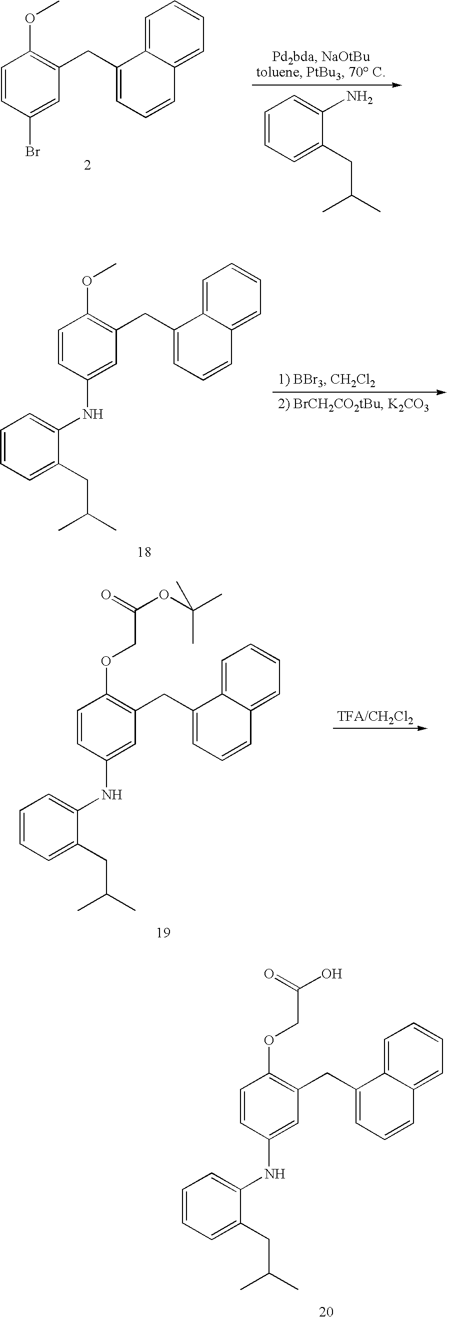

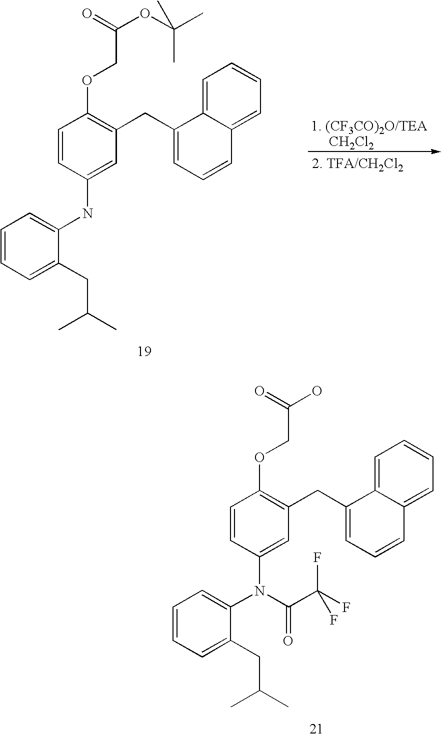

(2-Isobutyl-phenyl)-(4-methoxy-3-naphthalen-1-ylmethyl-phenyl)-amine, 18 In a 2 dram vial were placed the bromoanisole 2, (163 mg, 0.5 mmol), the alkyl aniline (72 mg, 0.5 mmol), tri-t-butylphosphine (90%, 5.0 mg, 0.014 mmol), Pd₂dba₃ (16.3 mg, 0.01 mmol), sodium t-butoxide (72 mg, 0.75 mmol) and toluene. The vial was flushed with nitrogen, capped and stirred magnetically at 70° C. overnight. The suspension was loaded directly onto a silica gel column and eluted with a 0 to 20% gradient of ethyl acetate in hexanes to afford 93 mg (47%) of the diphenyl amine as a colorless oil. ¹H NMR, 500 MHz, CDCl3 8.04-7.98(m, 1H), 7.88-7.83 (m, 1H), 7.737 (d, J=8.337,1H), 7.477 (ddd, J=4.042, 3.032, 1.011, 1H), 7.453 (ddd, J=4.295, 3.032, 1.263, 1H), 7.389 (dd, J=8.084, 7.074, 1H), 7.251 (d, J=7.074,1H), 7.07-6.73 (bm,5H), 6.591 (bs, 1H), 4.392 (s, 2H), 3.863 (bs, 3H), 2.349 (d, J=7.074, 2H), 1.835 (spt, J=6.568, 1H), 0.865 (d, J=6.316, 6H).

[4-(2-Isobutyl-phenylamino)-2-naphthalen-1-ylmethyl-phenoxy]-acetic acid tert-butyl ester, 19. In a 2 dram vial were placed the arylaminoanisole 18, (84 mg, 0.21 mmol) in dichloromethane (0.5 mL). BBr₃ (1M in dichloromethane, 1.0 mL) was added, the vial capped and the solution allowed to sit for 1.5 h. Water was added dropwise until heat evolution subsides. The mixture was partitioned between dichloromethane (5 mL) and water (3 mL). The organic layer was dried over Na₂SO₄. The solvents were removed and the residue dissolved in acetone (3 mL) in a 25 mL round-bottomed flask and treated with t-butyl bromoacetate (47 μL, 0.32 mmol) and K₂CO₃ (138 mg, 1 mmol). The reaction was brought to gentle reflux for 3 days after which time the solvents were removed. The residue was suspended in hexane and loaded directly onto a silica gel column and purified, eluting with a gradient from 0 to 10% ethyl acetate/hexane to afford a dark red solid. ¹H NMR (500 MHz CDCl₃) 8.029 (dd, J=6.316, 2.779, 1H), 7.86-7.82 (m, 1H), 7.728 (d, J=8.084, 1H), 7.49-7.44 (m, 2H), 7.379 (t, 7.831, 1H), 7.283 (d, J=6.821, 1H), 7.018 (d, J=6.568,1H), 6.97-6.76 (m,4H), 6.704 (d, J=7.831, 1H), 6.578 (s, 1H), 4.541 (s, 2H), 4.461 (s, 2H), 2.326 (d, J=7.074, 2H),1.782 (m, 1H), 1.491 (s, 9H), 0.832 (d, J=5.810, 6H).

[4-(2-Isobutyl-phenylamino)-2-naphthalen-1-ylmethyl-phenoxy]-acetic acid, 20. In a 2 dram vial were placed the t-butyl ester 19 (21.4 mg), followed by 20% (95% TFA/H₂O)/CH₂Cl₂ at which time the solution turned green. After 45 min sitting at room temperature, the solvent was removed by nitrogen stream. The red solid was purified on silica gel eluting with a gradient from ethyl acetate to 20% methanol/dichloromethane to afford 8.2 mg (42% yield) of a red solid. ¹H NMR (500 MHz CDCl₃) 8.018 (d, J=9.600, 1H), 7.87-7.84 (m, 1H), 7.744 (d, J=8.337, 1H), 7.49-7.45 (m, 2H), 7.386 (t, J=8.084, 1H), 7.257 (d, J=8.500, 1H), 7.042 (bs, 1H), 6.958 (bs, 2H), 6.864 (bs, 2H), 6.768 (bs, 1H), 6.609 (bs, 1H), 4.666 (bs 2H), 4.452 (bs, 2H), 2.348 (bs, 2H), 1.809 (spt, J=6.568, 1H), 0.850 (d, J=6.568, 6H); LCMS, (M+H) 439.6.

{4-[(2-Isobutyl-phenyl)-(2,2,2-trifluoro-acetyl)-aminol-2-naphthalen-1-ylmethyl-phenoxy}-acetic acid, 21. The diphenylaniline 19 (15 mg, 30 μmol) in dichloromethane was treated with trifluoroacetic anhydride (12 mg, 60 μmol) followed by triethylamine (10 μL, 70 μmol). After stirring overnight the solvents were removed and the residue purified on silica gel, eluting with a gradient from 0 to 20% ethyl acetate/hexane to afford 14 mg of the trifluoroacetamide. This product was dissolved in dichloromethane (1 mL) and treated with 0.5 mL 95% TFA/H₂O for 90 min after which time the solvents were removed and the residue purified on preparative LCMS, affording the final product in 25% yield over 2 steps.

### Example 5

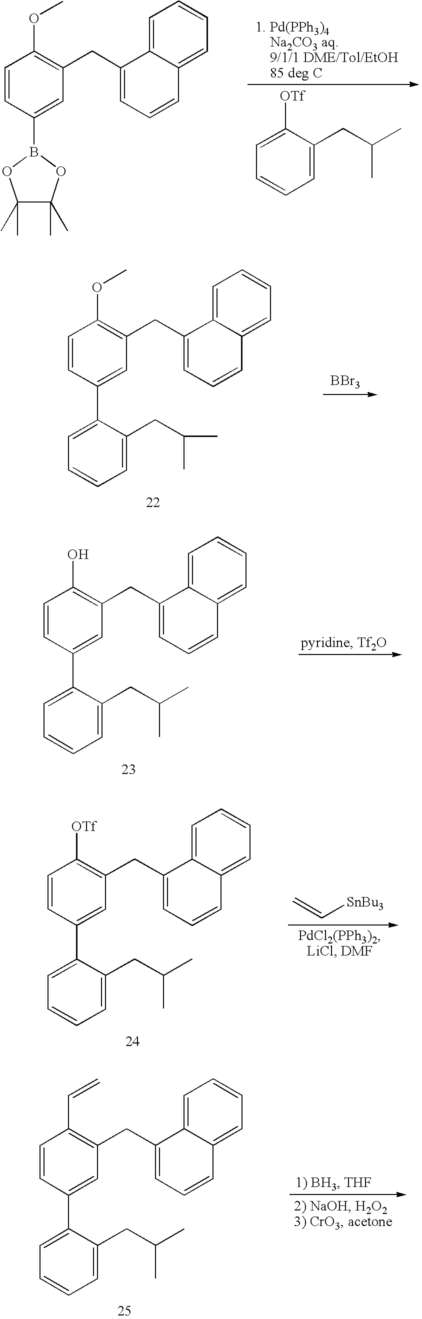

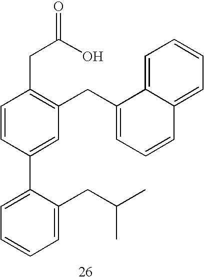

(2′-Isobutyl-4-methoxy-biphenyl-3-ylmethyl)-naphthalene, 22. In a 2 dram vial were placed the pinacol ester, (prepared as described for compound 4), (492 mg, 1.31 mmol), the aryl triflate (483 mg, 1.71 mmol), tetrakis(triphenylphosphine)palladium (378 mg, 0.33 mmol) and 9:1:1 DME/EtOH/Tol (4.4 mL). 2M Na₂CO₃ (1.31 mL) was added and the vial flushed with nitrogen and then tightly capped. The vial was stirred magnetically at 90° C. for 24 h after which time the solvents were removed by nitrogen stream and the residue partitioned between ethyl acetate and water. The organic layer was dried over Na₂SO₄ and purified on silica gel to afford 356 mg (71% yield) of the biphenyl. ¹H NMR (500 MHz, CDCl₃) 8.008 (dd, J=3.537, 6.316, 1H), 7.86-7.81 (m, 1H), 7.718 (d, J=8.337, 1H), 7.47-7.42 (m, 2H), 7.374 (dd, J=7.074, 8.084, 1H), 7.254 (d, J=9.000, 1H), 7.20-7.05 (m, 5H), 6.957 (d, J=8.337, 1H), 6.778 (d, J=2.274, 1H), 4.455 (s, 2H), 3.939 (s, 3H), 2.222 (d, J=7.074, 2H), 1.462 (spt, J=6.821, 1H), 0.541 (d, J=6.568, 6H).

2′-Isobutyl-3-naphthalen-1-ylmethyl-biphenyl-4-ol, 23. In a 40 mL vial were placed the methoxy biphenyl 22 (0.32 g, 0.84 mmol) in CH₂Cl₂ (1 mL). 1M BBr₃ in CH₂Cl₂ (1.60 mL) was added and the vial capped. After 40 min water was added dropwise. The organic layer was separated and dried over Na₂SO₄. The solvents were removed and the residue purified on silica gel, eluting with a gradient from 0-20% ethyl acetate/hexane to afford 214 mg (70%) ¹H NMR (500 MHz CDCl₃) 8.09-8.05 (m, 1H), 7.89-7.84 (m, 1H), 7.757 (d, J=8.084, 1H), 7.51-7.46 (m, 2H), 7.396 (dd, 7.074, 8.337, 1H), 7.286 (dd, J=0.758, 7.074, 1H), 7.22-7.094 (m, 4H), 7.045 (dd, J=2.021, 8.084, 1H), 6.918 (d, J=2.021, 1H), 6.867 (d, J=8.084, 1H), 4.477 (s, 2H), 2.314 (d, J=7.326, 2H), 1.525 (spt, J=6.568, 1H), 0.607 (d, J=6.821, 6H).

2′-Isobutyl-3-naphthalen-1-ylmethyl-biphenyl-4-ol, 24. In a 25 mL round-bottomed flask was placed pyridine (3 mL) and biphenol 23 (173 mg, 0.47 mmol) followed by trifluoromethanesulfonic anhydride (168 μL, 1 mmol). After stirring 30 min the solvents were evaporated. The residue was partitioned between water/CH₂Cl₂ and the organic layer dried over Na₂SO₄. The solvents were removed and the residue purified on silica gel, eluting with a gradient from 0-10% ethyl acetate/hexane to afford 88 mg (38% yield) of the triflate as a colorless oil. ¹H NMR(500 MHz, CDCl₃) 7.87-7.79 (m, 2H), 7.773 (d, J=8.337, 1H), 7.48-7.42 (m, 3H), 7.397 (d, J=6.821, 1H), 7.378 (d, J=7.326, 1H), 7.306 (d, J=6.568, 1H), 7.22-7.15 (m, 2H), 7.15-7.06 (m, 2H), 7.000 (dd, J=1.011, 7.326, 1H), 6.800 (d, J=2.021, 1H), 4.556 (s, 2H), 2.080 (d, 7.074, 2H), 1.341 (spt, J=6.821, 1H), 0.466 (d, J=6.568, 6H).

1-(2′-Isobutyl-4-vinyl-biphenyl-3-ylmethyl)-naphthalene, 25. In a 2 dram vial were placed biphenytriflate 24 (77 mg, 0.154 mmol), tributylvinyltin (68 mL, 0.23 mmol), DMF (1.0 mL), anhydrous LiCl (17.7 mg, 0.40 mmol) and dichlorobis(triphenylphosphine) palladium (12 mg, 0.018 mmol). The vial was flushed with nitrogen, capped tightly and heated to 50° C. overnight. The reaction mixture was partitioned between water and dichloromethane, the aqueous layer was extracted with dichloromethane (2×) and dried over Na₂SO₄. The solvents were removed and the residue purified on silica gel eluting with a gradient from 0 to 10% ethyl acetate/hexane to afford 55 mg(81%yield) as a colorless amorphous solid. ¹H NMR(500 MHz, CDCl₃) 8.038-8.003 (m, 1H), 7.882-7.854(m, 1H), 7.729(d, J=8.337, 1H), 7.642(d, J=8.084, 1H), 7.506(td, J=6.821, 2.021, 1H), 7.481(dd, J=6.821, 2.021, 1H), 7.348(dd, J=7.074, 8.337, 1H), 7.23-7.11(m, 5H), 7.050(dd, J=1.011, 7.074, 1H), 6.996(dd, J=17.431, 10.863, 1H), 6.993(d, J=1.516, 1H), 5.754(dd, J=1.516, 17.431, 1H), 5.289(dd, J=10.863, 1.263, 1H), 4.532(s, 2H), 2.327(d, J=7.326, 1H), 1.546(spt, J=6.821, 1H), 0.616(d, J=6.821, 6H).

(2′-Isobutyl-3-naphthalen-1-ylmethyl-biphenyl-4-yl)-acetic acid, 26. In a 2 dram vial were placed the biphenyl styrene 25 (28.1 mg, 0.075 mmol) and BH₃ as a 1M THF solution (100 μL) was added. The solution was stirred for 2 h after which time TLC (1:9 ethyl acetate/hexane) indicated consumption of the starting material. 30% Hydrogen peroxide (100 μL) and 2M NaOH (200 μL) were added. After TLC indicated completion of reaction the solvents were removed and the residue partitioned between dichloromethane and water. The organic layer was dried over Na₂SO₄ and the solvents removed to afford the crude alcohol. The residue was dissolved in acetone and treated with 5 drops of Jones Reagent (4M oxidation equivalent) after which time the solution became deep blue. After 3 h stirring, Jones reagent was added dropwise until the orange color remained for 30 min. Isopropanol was added until the orange color was gone. The solvents were removed and the residue partitioned between water and ethyl acetate. The aqueous layer was extracted with ethyl acetate (2×) and the combined organic layers dried over Na₂SO₄. The solvents were removed and the residue purified on silica gel eluting with a gradient from 0 to 100% ethyl acetate/hexane to afford 10.8 mg (35% yield) of the acid. 1H NMR (500 MHz, CD₃OD/CD₃COCD₃) 8.007(dd, J=6.821, 2.274, 1H), 7.884(dd, J=7.074, 2.021, 1H), 7.782(d, J=8.084, 1H), 7.49-7.44(m, 2H), 7.43-7.37(m, 2H), 7.233(d, J=6.316, 1H), 7.19-7.07(m, 4H), 7.003(dd, J=6.316, 1.768, 1H), 6.698(s, 1H), 4.559(s, 2H), 3.820(s, 2H), 2.215(dd, J=7.074, 2.516, 2H), 1.436(spt, J=5.810, 1H), 0.527(dd, J=2.021, 6.568, 6H).

### Example 6

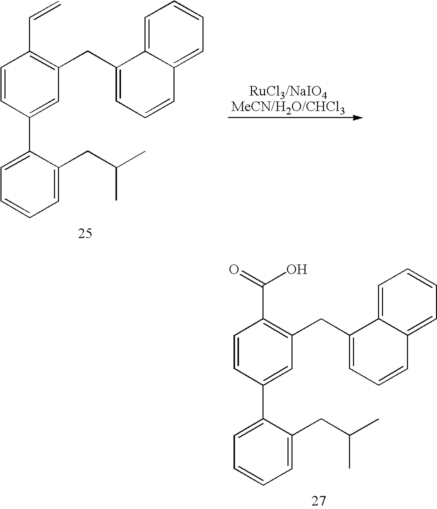

2′-Isobutyl-3-naphthalen-1-ylmethyl-biphenyl-4-carboxylic acid, 27. In a 2 dram vial were placed the biphenyl styrene 25 (20 mg, 0.053 mmol), acetonitrile (100 μL), water (200 μL), chloroform (100 μL), ruthenium(III)chloride (6.7 mg) and sodium metaperiodate (53 mg, 0.25 mmol). The reaction was stirred overnight after which time the solution was diluted with ethyl acetate (8 mL) and the solution dried over Na₂SO₄. The solution was filtered, the solvents evaporated and the residue purified on silica gel eluting with a gradient from 0-40% ethyl acetate/hexanes to afford the acid product (6.2 mg, 30% yield). ¹H NMR(500 MHz, CD₃OD/CD₃COCD₃) 8.091(d, J=8.084, 1H), 8.032 (dd, J=6.568, 3.284, 1H), 7.888(dd, J=3.537, 5.810, 1H), 7.774 (d, J=8.337, 1H), 7.486(t, J=3.284, 1H), 7.462(t, J=3.284, 1H), 7.402 (dd, J=7.074, 8.589, 1H), 7.285 (d, J=2.021, 1H), 7.229 (d, J=2.021, 1H), 7.201 (dd, J=1.768, 7.326, 1H), 7.17-7.12 (m, 2H), 7.030 (dd, J=1.768, 7.579, 1H), 6.900 (d, J=1.768, 1H), 4.966 (s, 2H), 2.201 (d, J=7.326, 2H), 1.386 (spt, J=6.568, 1H), 0.498 (d, J=6.568, 6H).

### Example 7

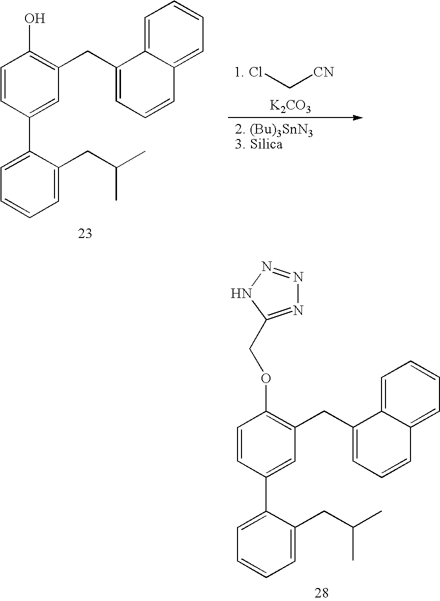

5-(2′-Isobutyl-3-naphthalen-1-ylmethyl-biphenyl-4-yloxymethyl)-1H-tetrazole, 28. To biphenol 23 (0.094 g, 0.26 mmol) in acetone (5 mL) was added chloroacetonitrile (165 μL, 2.6 mmol, 10 eqv.) and K₂CO₃ (106 mg, 0.77 mmol, 3 eqv.). The mixture was stirred under reflux conditions for 6 hrs., after which the solvent was removed under reduced pressure. The residue was partitioned between DCM and water, the organic layer was washed with brine and dried over Na₂SO₄. The solvent was evaporated and the crude material was purified by silica gel chromatography, using 0-20% EtOAc/hexanes.

To the compound obtained in the previous step (71 mg, 0.175 mmol) in 2 mL DME, (Bu)₃SnN₃ (0.1 mL, 0.35 mmol, 20 eqv.) was added. The reaction mixture was stirred at 85° C. overnight, was allowed to cool to rt and then poured into water. The compound was extracted into DCM, the organic layer was washed with water and dried over Na₂SO₄. Evaporation yielded the crude product, which was purified by column chromatography, using a hexanes/EtOAc 2/1 mixture.

### Example 8

### Solution for Parenteral Administration

A solution is prepared from the following ingredients:

| | |
|---|---|
| Active compound | 5 g |
| Sodium chloride for injection | 6 g |
| Sodium hydroxide for pH adjustment at pH | 5-7 |
| Water for inj. | Up to 1000 mL |

The active constituent and the sodium chloride are dissolved in the water. The pH is adjusted with 2M NaOH to pH 3-9 and the solution is filled into sterile ampoules.

### Example 9

Tablets for Oral Administration

1000 tablets are prepared from the following ingredients:

| | |
|---|---|
| Active compound | 100 g |
| Lactose | 200 g |
| Polyvinyl pyrrolidone | 30 g |
| Microcrystalline cellulose | 30 g |
| Magnesium stearate | 6 g |

The active constituent and lactose are mixed with an aqueous solution of polyvinyl pyrrolidone. The mixture is dried and milled to form granules. The microcrystalline cellulose and then the magnesium stearate are then admixed. The mixture is then compressed in a tablet machine giving 1000 tablets, each containing 100 mg of active constituent.

### Example 10

### Inhaler Powder

The active compound is micronized in a jet mill to a particle size suitable for inhalation (mass diameter<4 μm).

100 mg of the micronized powder is filled into a powder multidose inhaler (Turbohaler.RTM.). The inhaler is equipped with a dosing unit which delivers a dose of 1 mg.

### Example 11

The compounds in the following Table were synthesized using the methods described above; the structure of each compound was confirmed by ¹H NMR. Each compound also exhibited inhibitory activity in the NC-1 ELISA assay described above.

TABLE

| Com- | |
|---|---|
| pound No. | Structure |
| 5 | 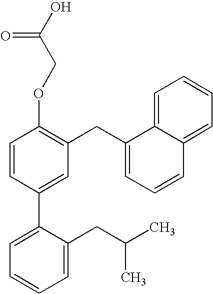 |
| 7 | 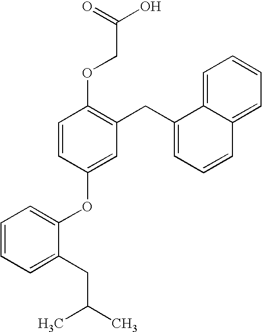 |
| 15 | 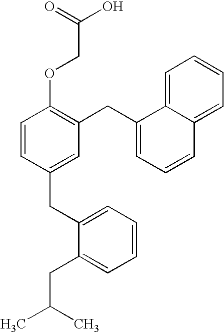 |
| 17 | 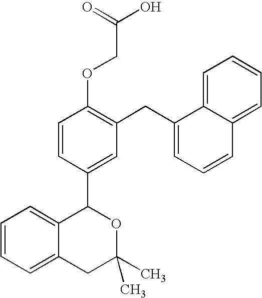 |
| 20 | 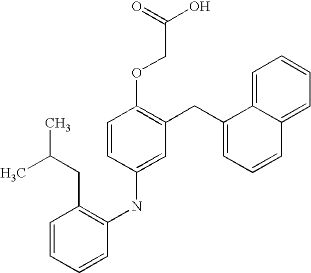 |
| 21 | 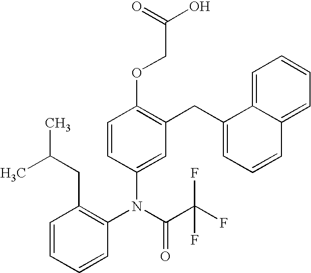 |
| 26 | 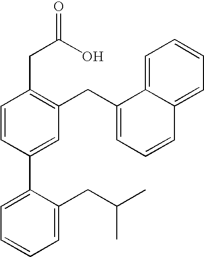 |
| 27 | 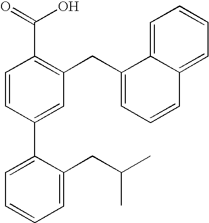 |
| 28 | 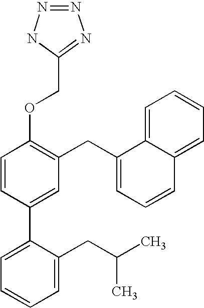 |
| 29 | 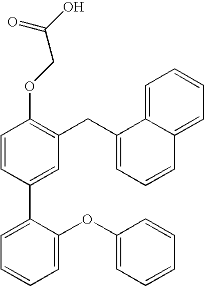 |
| 30 | 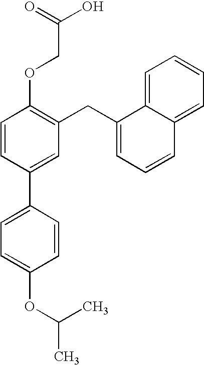 |
| 31 | 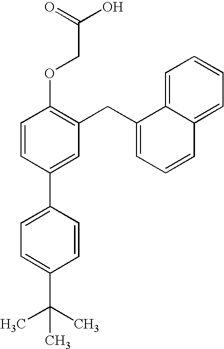 |
| 32 | 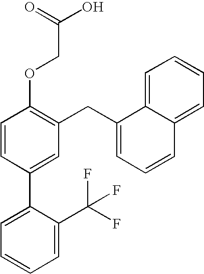 |
| 33 | 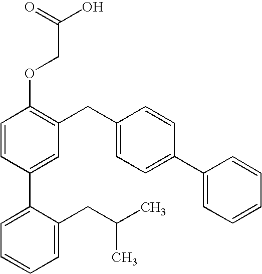 |
| 34 | 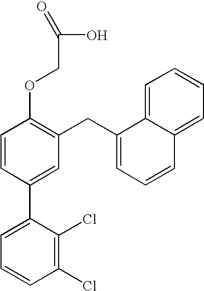 |
| 35 | 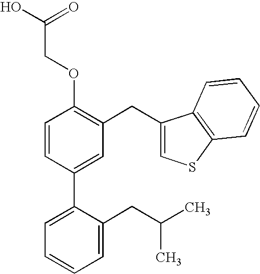 |
| 36 | 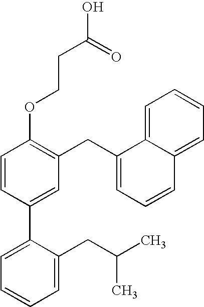 |
| 37 | 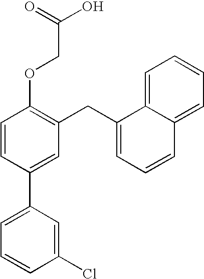 |
| 38 | 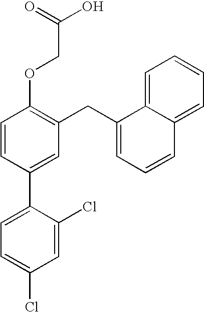 |
| 39 | 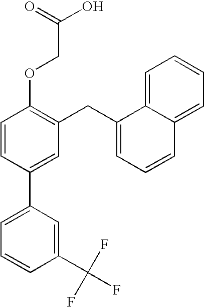 |
| 40 | 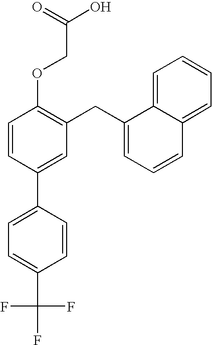 |
| 41 | 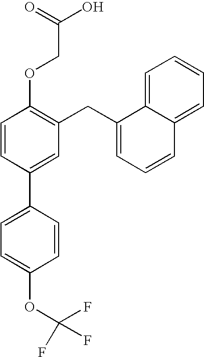 |
| 42 | 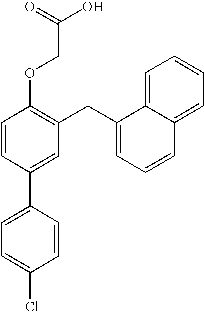 |
| 43 | 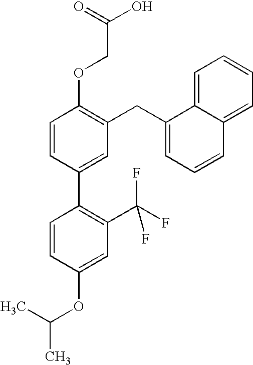 |
| 44 | 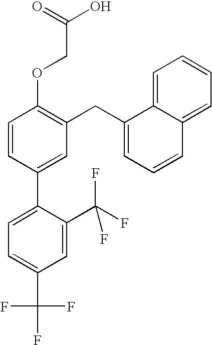 |
| 45 | 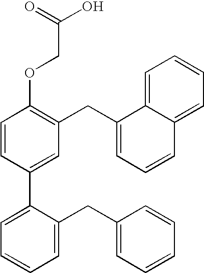 |
| 46 | 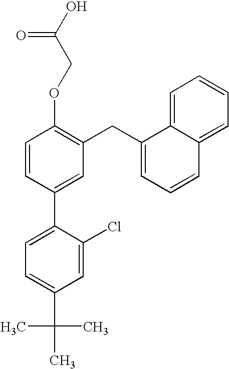 |
| 47 | 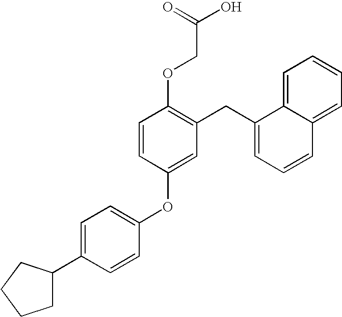 |
| 48 | 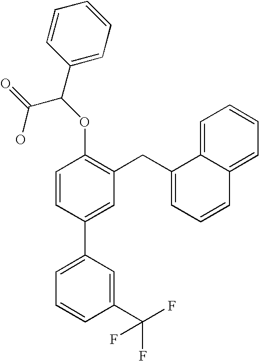 |
| 49 | 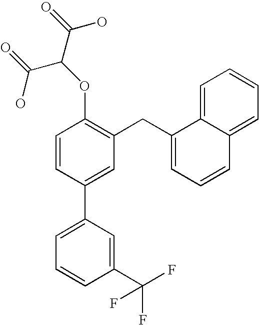 |
| 50 | 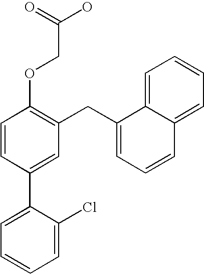 |
| 51 | 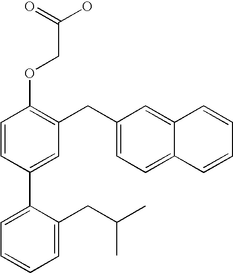 |
| 52 | 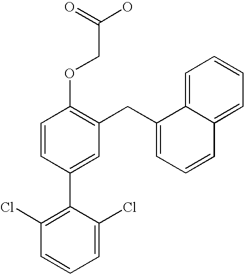 |
| 53 | 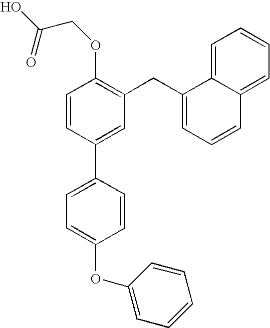 |
| 54 | 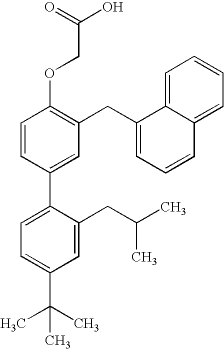 |
| 55 | 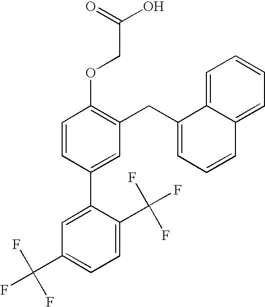 |
| 56 | 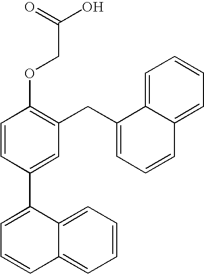 |
| 57 | 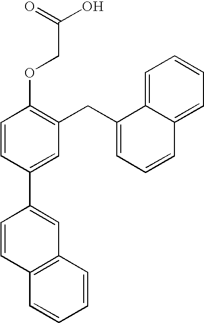 |
| 58 | 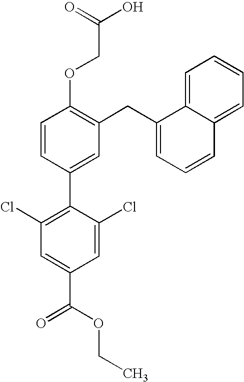 |
| 59 | 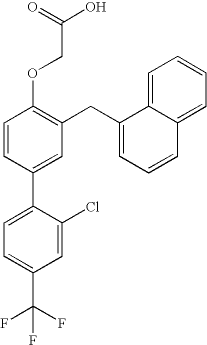 |
| 60 | 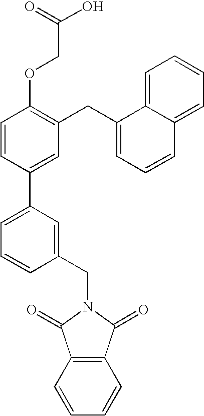 |
| 61 | 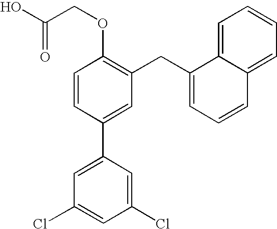 |
| 62 | 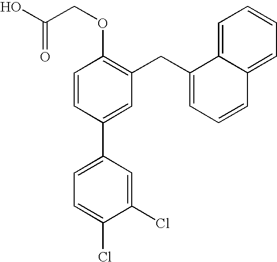 |
| 63 | 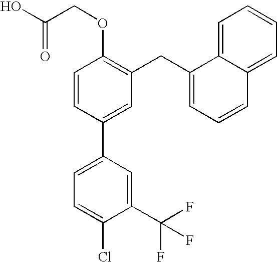 |
| 64 | 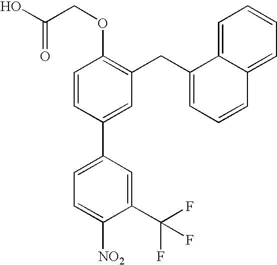 |
| 65 | 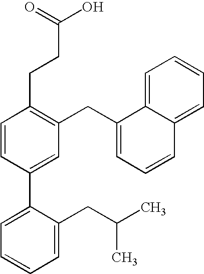 |
| 66 | 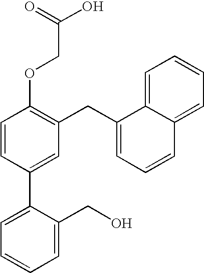 |
| 67 | 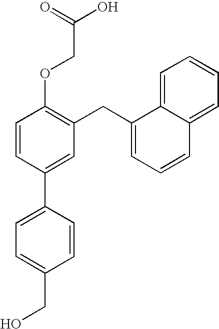 |
| 68 | 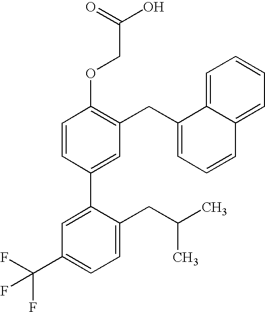 |
| 69 | 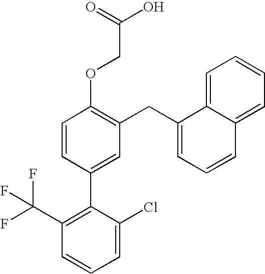 |
| 70 | 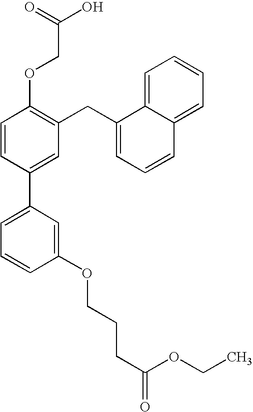 |
| 71 | 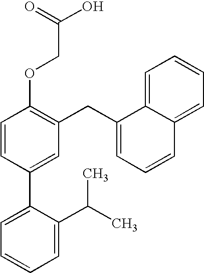 |
| 72 | 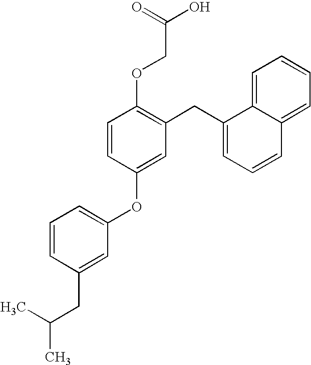 |
| 73 | 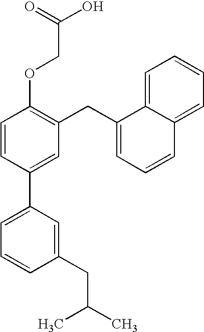 |
| 74 | 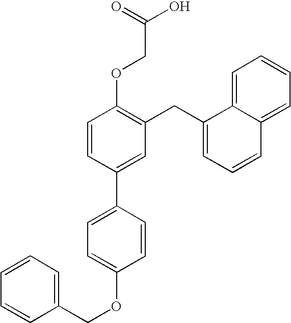 |
| 75 | 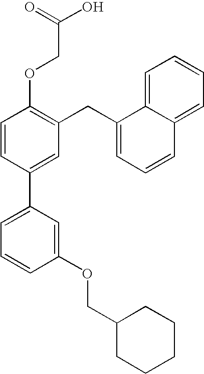 |
| 76 | 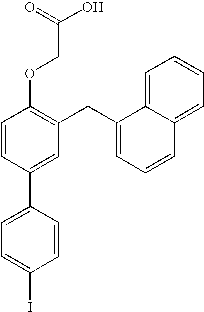 |
| 77 | 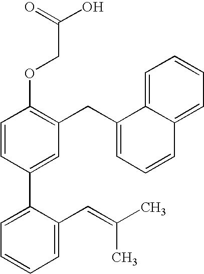 |
| 78 | 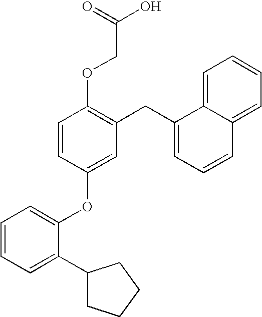 |
| 79 | 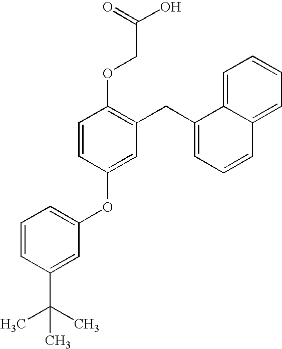 |
| 80 | 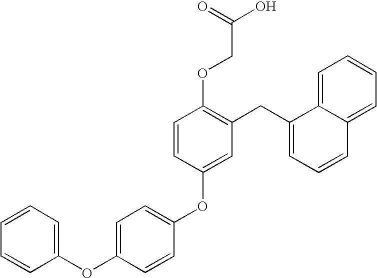 |
| 81 | 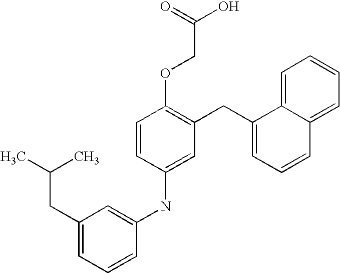 |
| 82 | 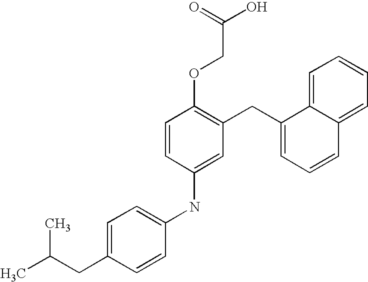 |

### Example 12

The anti-RSV activity of compounds of this invention is determined using an ELISA for F protein production (Huntley et al. Antimicrobial Agents and Chemotherapy 2002, 841-47). Vero or HFF cells are infected with virus and then incubated with inhibitor at different concentrations for four days. The inhibitory activity is assessed using an antibody to F protein to quantify viral proliferation. Compounds of this invention will be found to display activity in this assay.

### Example 13

Inhibition of influenza virus by compounds of the invention may be determined as follows. A viral plaque assay is performed according to the procedure of Kati et al. (Antimicrobial Agents and Chemotherapy 2002, 1014-21). Duplicate MDCK cell monolayers are inoculated with virus. After agitation for 1 hour the virus inoculum is discarded. The cell monolayers are overlaid with DMEM, agarose, trypsin, and inhibitor at different concentrations. After incubation for 72 hours the agar overlay is removed and the cell monolayers are stained. The antiviral efficacy is assessed by measuring the diameters of the plaques. Compounds of this invention will be found to display activity in this assay.

### Example 14

Inhibition of ebola virus by compounds of the invention may be determined as follows. A viral plaque assay is performed according to the procedure of Wilson et al. (Science 2000, 287, 1664-66). Inhibitor at varying concentrations is added to Vero cells that have been infected with virus. Cells are overlaid with agarose and incubated for six days. On the sixth day a second overlay is added that contains 5% neutral red. On the following day the plaques are counted. Compounds of this invention will be found to display activity in this assay.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A compound having a first planar moiety directly or indirectly attached to an acidic moiety, to a hydrophobic planar moiety, and to a second planar moiety bearing one or more non-aryl and non-heteroaryl substituents.

2. A compound having Formula I: 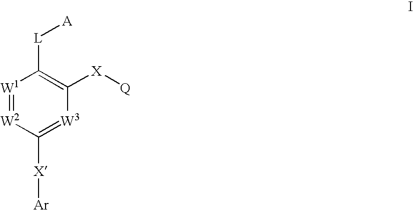
wherein:
A is hydrogen, OH, NO₂, —COOR, —C(O)NROH, —C(O)CF₃, —B(OH)₂, —SO₃H, —PO₃R₂, —OPO₃R₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR;
L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, —NR—C(O)NR-(CR⁴R⁵)ₘ—, —S(O)₂—NR—(CR⁴R⁵)ₘ—, or —NR—S(O)₂—(CR⁴R⁵)ₘ—, provided that L and A together are not H, —CH₃, OH, or —OCH₃;
W¹ is N or CR′;
W² is N or CR^{2;}
W³ is N or CR^{3;}
X is —(CR⁶R⁷)ᵣ—, —O—(CR⁶R⁷)ᵣ—, —S(O)_{q}—(CR⁶R⁷)ᵣ—, —NR—(CR⁶R⁷)ᵣ—, —NR—C(O)—(CR⁶R⁷)ᵣ—, —C(O)O—(CR⁶R⁷)ᵣ—, —C(O)NR—(CR⁶R⁷)ᵣ—, —NR—C(O)—O(CR⁶R⁷)ᵣ—, —NR—C(O)NR—(CR⁶R⁷)ᵣ—, —S(O)₂—NR—(CR⁶R⁷)ᵣ—, or —NR—S(O)₂—(CR⁶R⁷)ᵣ—;
X′ is a covalent bond, O, S(O)_{q}, NR, —N(C(O)—R)—, —N(C(O)—OR)—, —N(C(O)—NRR)—, —NR—C(O)—, —NR—C(O)—NR—, substituted or unsubstituted C₁₋₄ alkyl, substituted or unsubstituted C₂ alkenyl, or acetylenyl;
Q is a substituted or unsubstituted cycloalkyl, substituted or unsubstituted cycloalkenyl, substituted or unsubstituted aryl, substituted or unsubstituted aralkyl, substituted or unsubstituted heterocyclyl, or substituted or unsubstituted heterocyclylalkyl;
Ar is aryl or heterocyclyl, each substituted with one or more R′;
R at each occurrence is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted (C₀₋₄ alkylene)(C₆₋₁₀ aryl), or substituted or unsubstituted (C₀₋₄ alkylene)(C₁₋₉ heterocyclyl);
R′ at each occurrence is independently, F, Cl, Br, I, NO₂, CN, substituted or unsubstituted C₁₋₈ alkyl, substituted or unsubstituted C₂₋₈ alkenyl, substituted or unsubstituted (C₁₋₆ alkylene)(C₆₋₁₄ aryl), substituted or unsubstituted (C₁₋₆ alkylene)(C₁₋₁₃ heterocycl), OR⁸, —C(O)R⁸, —COOR⁸, —S(O)_{q}R⁸, —NR⁸R⁹, —C(Y)NR⁸R⁹, —N(R⁸)C(Y)OR⁹, —NR¹⁰C(Y)NR⁸R⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹, wherein Y is O or S;
R¹, R², and R³, at each occurrence, are independently hydrogen, F, Cl, Br, I, CN, NO₂, substituted or unsubstituted C₁₋₈ alkyl, substituted or unsubstituted C₂₋₈ alkenyl, substituted or unsubstituted (C₀₋₆ alkylene)(C₆₋₁₄ aryl), substituted or unsubstituted (C₀₋₆ alkylene)(C₁₋₁₃ heterocyclyl), OR⁸, —C(O)R⁸, —COOR⁸, —S(O)_{q}R⁸, —NR⁸R⁹, —C(Y′)NR⁸R⁹, —N(R⁸)C(Y′)OR⁹, —NR¹⁰C(Y′)NR⁸R⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹, wherein each Y′ is independently O or S;
R⁴ and R⁵ are, at each occurrence, independently hydrogen, F, Cl, Br, I, substituted or unsubstituted straight or branched C₁₋₄ alkyl, substituted or unsubstituted C₂₋₄ alkenyl, substituted or unsubstituted aryl, substituted or unsubstituted heterocyclyl, —OR, —COOR —NRR; or R⁴ and R⁵, together with the carbon to which they are attached, form a carbonyl;
R⁶ and R⁷ are, at each occurrence, independently hydrogen, F, Cl, Br, I, substituted or unsubstituted straight or branched C₁₋₄ alkyl, substituted or unsubstituted C₂₋₄ alkenyl, —OR, —COOR —NRR; or when r is 2 or 3, R⁶ and R⁷, together with the carbon to which they are attached, may form a carbonyl;
R⁸, R⁹, R¹⁰, and R¹¹, at each occurrence, are independently hydrogen, substituted or unsubstituted C₁₋₈ alkyl, substituted or unsubstituted C₂₋₆ alkenyl, substituted or unsubstituted (C₀₋₆ alkylene)(C₆₋₁₀ aryl), or substituted or unsubstituted (C₀₋₆ alkylene)(C₁₋₉ heterocyclyl); or R⁸ and R⁹, together with the N to which they are attached, form a substituted or unsubstituted heterocyclic ring;
m=0-4;
each q is independently 0-2; and
r=0-3;
and stereoisomers thereof, tautomers thereof, solvates thereof, prodrugs thereof, and pharmaceutically acceptable salts thereof;
provided the compound is not acetic acid 3′-(2-acetoxy-4-methoxy-benzoyl)-5-benzoyl-2-methoxy-biphenyl-4-yl ester, acetic acid 5′-(2-acetoxy-4-methoxy-benzoyl)-2,2′-dimethoxy-5-(4-methoxy-benzoyl)-biphenyl-4-yl ester, 5,5′-bis-[bis-(4-tert-butyl-phenyl)-methoxy-methyl]-2,4,2′,4′-tetraisopropyl-biphenyl, 3-acetoxy-5-methyl-2-[2,4,2′,4′-tetraacetoxy-3′-(2-methoxycarbonyl-4-methyl-6-acetoxybenzoyl)-biphenyl-3-carbonyl]-benzoic acid methyl ester, 3-(3-benzyl-4′-methoxy-biphenyl-4-yl)-propionic acid, 3-(3-benzyl-4′-methoxy-biphenyl-4-yl)-propionyl chloride, or (4,4′-diamino-3′-benzoyl-biphenyl-3-yl)-phenyl-methanone.

3. The compound of claim 2, wherein A is OH, NO₂, —COOR, —C(O)NROH, —C(O)CF₃, —B(OH)₂, —SO₃H, —PO₃H₂, —OPO₃H₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR.

4. The compound of claim 2, wherein A is hydrogen, —COOR, —C(O)NROH, —C(O)CF₃, —B(OH)₂, —SO₃H, —PO₃H₂, —OPO₃H₂, or substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R, —COOR, —C(O)NRR, —OC(O)R, —NRC(O)R, —OC(O)NR, and —NRC(O)OR.

5. The compound of claim 2, wherein A is substituted or unsubstituted tetrazole, triazole, thiazole, oxazole, isoxazole, imidazole, or pyrazole, wherein the substituents are selected from the group consisting of F, Cl, Br, I, OR, CN, NRR, NO₂, R. —COOR, —C(O)NRR, —OC(O)R, —NRC(O)R, —OC(O)NR, and —NRC(O)OR.

6. The compound of claim 2, wherein A is —COOR, —C(O)NHOH, —C(O)CF₃, or —B(OH)₂.

7. The compound of claim 2, wherein A is —COOR.

8. The compound of claim 2, wherein A is —COOH.

9. The compound of claim 2, wherein L is —(CR⁴R⁵)ₘ—, —O—(CR⁴R⁵)ₘ—, —S(O)_{q}—(CR⁴R⁵)ₘ—, —NR—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—.

10. The compound of claim 2, wherein L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, or —C(O)NR—(CR⁴R⁵)ₘ—.

11. The compound of claim 2, wherein L is —(CR⁴R⁵)ₘ—, —O—(CR⁴R⁵)ₘ—, —S(O)_{q}—(CR⁴R⁵)ₘ—, or —NR—(CR⁴R⁵)ₘ—.

12. The compound of claim 2, wherein L is —(CR⁴R⁵)_{m—} or —O—(CR⁴R⁵)ₘ—.

13. The compound of claim 2, wherein L is —O—(CR⁴R⁵)ₘ—.

14. The compound of claim 13, wherein R⁴ and R⁵ are each hydrogen.

15. The compound of claim 13, wherein m=1-2.

16. The compound of claim 2, wherein L and A together are —CR⁴R⁵)ₘ—COOR or —O—(CR⁴R⁵)ₘ—COOR.

17. The compound of claim 2 wherein R⁴ and R⁵ are, at each occurrence, independently hydrogen, F, Cl, Br, I, substituted or unsubstituted straight or branched C₁₋₄ alkyl, substituted or unsubstituted C₂₋₄ alkenyl, OR, COOR, or —NRR; or R⁴ and R⁵, together with the carbon to which they are attached, form a carbonyl.

18. The compound of claim 2, wherein m=1-3.

19. The compound of claim 2, wherein X is —(CR⁶R⁷)ᵣ—, —O—(CR⁶R⁷)ᵣ—, —S(O)_{q}—(CR⁶R⁷)ᵣ—, —NR—(CR⁶R⁷)ᵣ—, —NR—C(O)O—(CR⁶R⁷)ᵣ—, —C(O)NR—(CR⁶R⁷)ᵣ—, —NR—C(O)—O(CR⁶R⁷)ᵣ—, or —NR—C(O)NR—(CR⁶R⁷)ᵣ—.

20. The compound of claim 2, wherein X is —(CR⁶R⁷)ᵣ—, —O—(C R⁶R⁷)ᵣ—, —S(O)_{q}—(CR⁶R⁷)ᵣ—, —NR—(CR⁶R⁷)ᵣ—, —C(O)O—(CR⁶R⁷)ᵣ—, or —C(O)NR—(CR⁶R⁷)ᵣ—.

21. The compound of claim 2, wherein X is —(CR⁶R⁷)ᵣ—, —O—(C R⁶R⁷)ᵣ—, or —S(O)_{q}—(CR⁶R⁷)ᵣ—.

22. The compound of claim 2, wherein X is —(CR⁶R⁷)ᵣ—.

23. The compound of claim 22, wherein X is —CH₂—.

24. The compound of claim 2, wherein Q is a substituted or unsubstituted cycloalkyl or substituted or unsubstituted cycloalkenyl.

25. The compound of claim 2, wherein Q is a substituted or unsubstituted aryl, substituted or unsubstituted aralkyl, substituted or unsubstituted heterocyclyl, or substituted or unsubstituted heterocyclylalkyl.

26. The compound of claim 2, wherein Q is a substituted or unsubstituted aryl or substituted or unsubstituted aralkyl.

27. The compound of claim 2, wherein Q is a fused or unfused bicyclic ring selected from the group consisting of substituted and unsubstituted C₉₋₁₂ aryl, substituted and unsubstituted C₇₋₁₂ cycloalkyl, substituted and unsubstituted C₉₋₁₂ cycloalkenyl, and substituted and unsubstituted C₇₋₁₂ heterocyclyl.

28. The compound of claim 2, wherein Q is a fused or unfused bicyclic ring that is substituted or unsubstituted C₉₋₁₂ aryl.

29. The compound of claim 2, wherein Q is substituted or unsubstituted 1-naphthyl, 2-naphthyl, or 4-biphenyl.

30. The compound of claim 29, wherein X is —CH₂—.

31. The compound of claim 2 wherein X′ is a covalent bond, O, S(O)_{q}, —NR—, —NR—C(O)—, —NR—C(O)—NR—, substituted or unsubstituted C₁₋₂ alkyl, substituted or unsubstituted C₂ alkenyl, or acetylenyl.

32. The compound of claim 2 wherein X′ is a covalent bond, O, S(O)_{q}, or —NR—.

33. The compound of claim 2 wherein X′ is a substituted or unsubstituted C₁₋₂ alkyl.

34. The compound of claim 2 wherein X′ is a covalent bond.

35. The compound of claim 2 wherein X′ is —N(C(O)—R)—, —N(C(O)—OR)—, or —N(C(O)—NRR)—.

36. The compound of claim 2, wherein W¹ is CR¹.

37. The compound of claim 2, wherein W² is CR².

38. The compound of claim 2, wherein W³ is CR³.

39. The compound of claim 2, wherein W¹ is CR¹, W² is CR², and W³ is CR³.

40. The compound of claim 2, wherein W¹ is N, W² is N, and W³ is CR³.

41. The compound of claim 2, wherein W¹ is CR¹, W² is N, and W³ is N.

42. The compound of claim 2, wherein Ar is a 6-member aryl, a 5-or 6-member heteroaryl, a 9-12 member bicyclic aryl or heterocyclyl, each substituted with one or more R′.

43. The compound of claim 2, wherein Ar is a 6-member aryl or a 5-, or 6-member heteroaryl, each substituted with one or more R′.

44. The compound of claim 2, wherein Ar is a 9-12 member bicyclic aryl or heterocyclyl, each substituted with one or more R′.

45. The compound of claim 2, wherein Ar is 6-member aryl, substituted with one or more R′.

46. The compound of claim 2, wherein Ar is a 5- or 6-member heteroaryl, substituted with one or more R′.

47. The compound of claim 2, wherein Ar is substituted with one or more R′ and is selected from the group consisting of phenyl, pyrrolyl, imidazolyl, pyrazolyl, triazolyl, furanyl, thiophenyl, oxazolyl, isooxazolyl, oxadiazolyl, thiazolyl, isothiazolyl, thiadiazolyl, pyridinyl, pyridazinyl, pyrimidinyl, pyrazinyl, and triazinyl.

48. The compound of claim 2, wherein Ar is substituted with one or more R′ and is selected from the group consisting of phenyl, pyrrolyl, imidazolyl, pyrazolyl, furanyl, thiophenyl, oxazolyl, isooxazolyl, thiazolyl, isothiazolyl, pyridinyl, pyridazinyl, pyrimidinyl, and pyrazinyl.

49. The compound of claim 2, wherein Ar is substituted with one or more R′ and is selected from naphthyl, indolyl, benzofuranyl, benzthiazolyl, benzothiophenyl, chromanyl, isochromanyl, or coumarinyl.

50. The compound of claim 2, wherein Ar is phenyl substituted with one or more R′.

51. The compound of claim 2, wherein R¹, R², and R³, at each occurrence, are independently hydrogen, F, Cl, Br, I, CN, NO₂, substituted or unsubstituted C₁-C₈ alkyl, substituted or unsubstituted C₂₋₈ alkenyl, substituted or unsubstituted (C₀₋₆ alkylene)(C₆₋₁₄ aryl), substituted or unsubstituted (C₀₋₆ alkylene)(C₁₋₁₃ heterocyclyl), —OR⁸, —C(O)R⁸, —COOR⁸, —S(O)_{q}R⁸, —NR⁸R⁹, —C(O)NR⁸R⁹, —N(R⁸)C(O)OR⁹, —NR¹⁰C(O)NR⁸R⁹, —NR¹⁰C(NR¹¹)NR⁸R⁹, —C(NR¹⁰)NR⁸R⁹, —NR¹⁰NR⁸R⁹, —NR⁸OR⁹, —S(O)_{q}NR⁸R⁹, or —NR⁸—SO₂—R⁹.

52. The compound of claim 2, wherein R′, at each occurrence, is independently F, Cl, Br, I, CN, NO₂, substituted or unsubstituted C₁₋₈ alkyl, substituted or unsubstituted C₂₋₈ alkenyl, substituted or unsubstituted (C₁₋₆ alkylene)(C₆₋₁₄ aryl), substituted or unsubstituted (C₁₋₆ alkylene)(C₁₋₁₃ heterocyclyl), —OR⁸, —C(O)R⁸, —COOR⁸, —NR⁸R⁹, —C(Y)NR⁸R⁹, or —N(R⁸)C(Y)OR⁹, wherein Y is O or S.

53. The compound of claim 2 wherein R′, at each occurrence, is independently F, Cl, Br, I, NO₂, substituted or unsubstituted C₁₋₈ alkyl, substituted or unsubstituted C₂₋₈ alkenyl, OR⁸, or —COOR⁸.

54. The compound of claim 2, wherein R⁸ and R⁹, together with the nitrogen to which they are attached, form a substituted or unsubstituted heterocyclyl.

55. The compound of claim 54, wherein the heterocyclyl is selected from the group consisting of pyrrolidinyl, piperidinyl, morpholinyl, and pyrazinyl.

56. The compound of claim 2 having Formula V 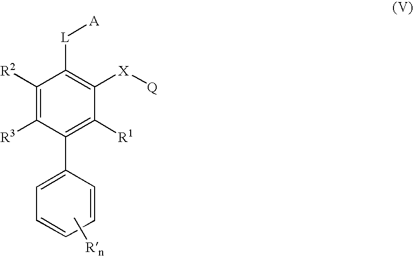
wherein n=1-5.

57. The compound of claim 56 wherein A is hydrogen, —COOR, —C(O)NROH, —C(O)CF₃, —B(OH)₂, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR.

58. The compound of claim 56, wherein L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—.

59. The compound of claim 56, wherein L is —(CR⁴R⁵)ₘ— or —O—(CR⁴R⁵)ₘ—.

60. The compound of claim 56, wherein L and A together are —(CR⁴R⁵)ₘ—COOR or —O—(CR⁴R⁵ )ₘ—COOR.

61. The compound of claim 2 having Formula VI 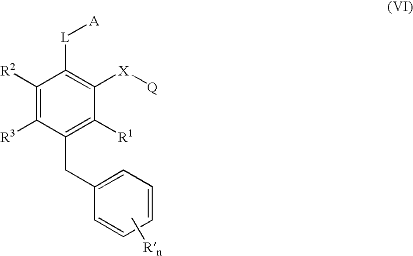
wherein n=1-5.

62. The compound of claim 61 wherein A is hydrogen, —COOR, —C(O)NROH, —C(O)CF₃, —B(OH)₂, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR.

63. The compound of claim 61, wherein L is —(CR⁴R⁵)ₘ—, —O—(CR⁴R⁵)ₘ—, —S(O)_{q}—(CR⁴R⁵)ₘ—, —NR—(CR⁴R⁵)ₘ—, —NR—C(O)—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, or —NR—C(O)NR—(CR⁴R⁵)ₘ—.

64. The compound of claim 61, wherein L is —(CR⁴R⁵)ₘ— or —O—(CR⁴R⁵)ₘ—.

65. The compound of claim 61, wherein L and A together are —(CR⁴R⁵)ₘ—COOR or —O—(CR⁴R⁵)ₘ—COOR.

66. A pharmaceutical composition, comprising a pharmaceutically effective amount of the compound of claim 2 and a pharmaceutically acceptable carrier or diluent.

67. A method for the treatment of viral infection, the method comprising administering the composition of claim 66 to a subject in need thereof.

68. The method of claim 67, wherein the viral infection is HIV, ebola, HRSV, or influenza infection.

69. The method of claim 67, wherein the viral infection is HIV

70. A method for the inhibition of cell entry by a virus, the method comprising contacting a virus with a compound of claim 2.

71. The method of claim 70, wherein the virus is HIV, ebola, HRSV, or influenza.

72. The method of claim 70, wherein the virus is HIV.

73. A method of preparing a compound of claim 2 wherein X′ is a covalent bond or NH, the method comprising
reacting a compound of Formula III 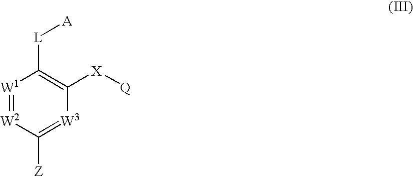
with a compound of Formula IV
Z′-Ar (IV)
in the presence of a palladium catalyst, a base, and a solvent
to form a compound of claim 2 wherein X′ is a covalent bond or NH, and wherein
A, Ar, L, X, Q, Z, W¹, W², and W³ are as defined in claim 2;
Z is B(OR″)₂ or NH₂, and Z′ is I, Br, Cl, or OTf; or
Z is I, Br, Cl, or OTf, and Z′ is B(OR″)₂ or NH₂; and
wherein each R″ is independently hydrogen or substituted or unsubstituted alkyl, or, each R″, together with B and the atoms to which they are attached, form a cyclic boronate.

74. The method of claim 73, wherein the palladium catalyst is Pd₂(dba)₃ or Pd(PPh₃)₄.

75. The method of claim 73, wherein the base is Na₂CO₃, K₂CO₃, or NaOtBu.

76. The method of claim 73, wherein the solvent is DMF, toluene, or a mixture of DME, ethanol and toluene.

77. A method of preparing a compound of claim 2 wherein X′ is O, the method comprising
reacting a compound of Formula III 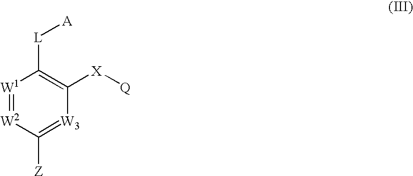
with a compound of Formula IV
Z′-Ar (IV)
in the presence of a copper catalyst, a base, and a solvent
to form a compound of claim 2 wherein X′ is O, and wherein
A, Ar, L, X, Q, Z, W¹, W², and W³ are as defined in claim 2;
Z is OH, and Z′ is I, Br, Cl, or OTf; or Z is I, Br, Cl, or OTf, and Z′ is OH.

78. The method of claim 77, wherein the copper catalyst is Cul.

79. The method of claim 77, wherein the base is Cs₂CO₃.

80. The method of claim 77, wherein the solvent is toluene.

81. A method of preparing a compound of claim 2 wherein X′ is —CH(OH)—, the method comprising
reacting a compound of Formula III 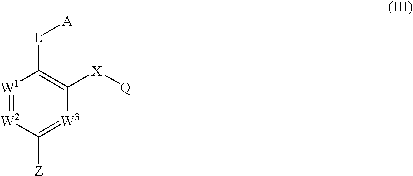
with a compound of Formula IV
Z′-Ar (IV)
in the presence of a solvent
to form a compound of claim 2 wherein X′ is —CH(OH)—, and wherein
A, Ar, L, X, Q, W¹, W², and W³ are as defined in claim 2;
Z is Li, and Z′ is C(O)—H; or Z is C(O)—H, and Z′ is Li.

82. The method of claim 81, wherein the solvent is THF or diethylether.

83. A method of preparing a compound of claim 2, wherein X′ is —CH₂—, the method comprising treating a compound having Formula VII 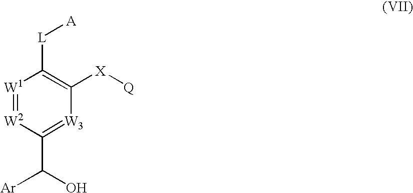
with a reducing agent in a solvent.

84. The method of claim 83 where the reducing agent is H₂ in the presence of Pd/C or triethylsilane with trifluoroacetic acid.

85. The method of claim 83, where the solvent is EtOAc or DCM.

86. An intermediate having the Formula III: 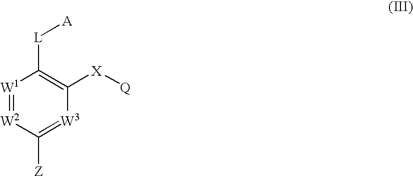
wherein,
A is hydrogen, OH, NO₂, —COOR, —C(O)NROH, —C(O)CF₃, —B(OH)₂, —SO₃H, —PO₃R₂, —OPO₃R₂, —C(O)NHSO₂R, or substituted or unsubstituted tetrazole, triazole, thiazole, oxazole, isoxazole, imidazole, or pyrazole, wherein the substituents are selected from the group consisting of F, Cl, Br, I, —OR, —CN, —NRR, —NO₂, —R, —COOR, —C(O)NRR, —OC(O)R, —NRC(O)R, —OC(O)NR, and —NRC(O)OR;
L is —(CR⁴R⁵)ₘ—, —O—(CR⁴R⁵)ₘ—, —S(O)_{q}—(CR⁴R⁵ )ₘ—, —NR—(CR⁴R⁵)ₘ—, —C(O)O—(CR⁴R⁵)ₘ—, —C(O)NR—(CR⁴R⁵)ₘ—, —NR—C(O)—O(CR⁴R⁵)ₘ—, —NR—C(O)NR—(CR⁴R⁵)ₘ—, —S(O)₂—NR—(CR⁴R⁵)ₘ—, or —NR—S(O)₂—(CR⁴R⁵)ₘ—;
W¹ is N or CR¹;
W² is N or CR²;
W³ is N or CR³;
X is —(CR⁶R⁷)ᵣ—, —O—(CR⁶R⁷)ᵣ—, —S(O)_{q}—(CR⁶R⁷)ᵣ—, —NR—(CR⁶R⁷)ᵣ—, —C(O)O—(CR⁶R⁷)ᵣ—, —C(O)NR—(CR⁶R⁷)ᵣ—, —NR—C(O)—O(CR⁶R⁷)ᵣ—, —NR—C(O)NR—(CR⁶R⁷)ᵣ—, —S(O)₂—NR—(CR⁶R⁷)ᵣ—, or —NR—S(O)₂—(CR⁶R⁷)ᵣ—;
Q is a substituted or unsubstituted cycloalkyl, substituted or unsubstituted cycloalkenyl, substituted or unsubstituted aryl, substituted or unsubstituted aralkyl, substituted or unsubstituted heterocyclyl, or substituted or unsubstituted heterocyclylalkyl;
Z is B(OR″)₂, NH₂, OH, I, Br, Cl, C(O)—H, Li or OTf;
wherein each R″ is independently hydrogen or substituted or unsubstituted alkyl, or, each R″ together with B and the atoms to which they are attached, form a cyclic boronate;
R at each occurrence is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted (C₀₋₄ alkylene)(C₆₋₁₀ aryl), or substituted or unsubstituted (C₀₋₄ alkylene)(C₁₋₉ heterocyclyl);
m=0-4;
each q is independently 0-2;
r=0-3;
and stereoisomers thereof, tautomers thereof, and solvates thereof.
